(19) United States
(12) Patent Application Publication
(10) Pub. No.: US 2023/0317204 A1
(43) Pub. Date: Oct. 5, 2023

(54) CELL-TYPE IDENTIFICATION

(71) Applicant: Hoffmann-La Roche Inc., Little Falls, NJ (US)

(72) Inventors: Wei-Yi CHENG, Little Falls, NJ (US); Tai-Hsien OU YANG, Little Falls, NJ (US); James Jinsong CAI, Little Falls, NJ (US)

(21) Appl. No.: 17/922,342

(22) PCT Filed: Apr. 29, 2021
(86) PCT No.: PCT/EP2021/061350

§ 371 Date: Oct. 28, 2022

(30) Foreign Application Priority Data
Apr. 30, 2020 (EP) 20172524.9

(51) Int. Cl. G16B 20/00; G16B 25/10; G16B 40/20

(52) U.S. Cl. CPC G16B 20/00; G16B 25/10; G16B 40/20

(57) ABSTRACT

The present invention provides a method comprising (a) obtaining a single cell gene expression profile comprising gene expression measurements for a set of genes, for a plurality of cells, and a single cell protein expression profile comprising protein expression measurements for two or more proteins, for the plurality of cells; (b) using the single cell protein expression profiles and an unsupervised learning method to assign a cell type class to at least some of the plurality of cells; and(d) applying a feature selection process to the single gene expression profiles to identify genes in the single cell gene expression profiles that are predictive of the cell type classes assigned in step (b), wherein the genes identified in step (d) form a predictor set of genes for predicting the cell type of one or more cells.

### FIELD OF THE INVENTION

The present invention relates to methods for identifying the cell type of a cell from a gene expression profile, to methods for defining predictor gene panels for use in such methods, and to related systems and devices.

### BACKGROUND TO THE INVENTION

Single-cell transcriptomics technologies such as single cell RNA sequencing (scRNA-seq) provide an opportunity to overcome the challenges in studying the unique functions of individual cells in heterogeneous biological specimen, greatly enhancing our ability to understand human diseases and the mechanisms of action of new drug candidates and biomarkers in drug development.

Accurate assignment of the types of the cells in a specimen is a critical step in any single cell data analysis. Traditionally, immunostaining techniques of the cell-type-specific surface marker proteins have been used to profile the composition of a specimen. Such cell type assignments, which can be obtained using technologies such as flow cytometry combined with labelled antibodies targeting surface marker proteins, are typically considered as a “gold standard”. However, these methods are typically low throughput (at least in terms of the single cell transcriptomics data that can be obtained from cell-typed samples) and require a priori knowledge about the epitopes of cell surface markers (Heath et al., 2016). In addition, immunostaining markers for cell type assignment are typically restricted to those that are expressed on the cell surface, limiting the genes that can be used as cell type markers.

In recent years, scRNA-seq has emerged as a new technology that provides high-throughput expression profiles of individual cells. Studies have shown that there is usually a lack of correlation between the transcript and protein levels of the cell surface markers commonly used to profile immune cells (Peterson et al., 2017; Stoeckius et al., 2017). Therefore, the information obtained at the transcript level cannot be used as a direct equivalent of the information traditionally used at the protein level.

Multiple methods for cell-type assignment using scRNA-seq data have been proposed (Abdelaal et al., 2019). A common strategy for modelling the cellular heterogeneity in scRNA-seq data relies on clustering approaches using the mRNA expression levels of pre-defined, curated panels of cell-type markers to assign cell types to clusters (see e.g. CellAssign—described in A. W. Zhang et al., 2019; Domanskyi et al., 2019; Ma et al., 2019; Xie et al., 2019; X. Zhang et al., 2019). Other published methods include SingleR (Aran et al., 2019), which assigns cell types by studying the correlation between the expression profile of a cell and reference expression profiles from previous experiments using pre-sorted cells, and CaSTLe (Lieberman et al., 2018) which assigns cell types using a machine learning model trained using RNA-seq data that has been manually curated to assign cell type labels to clusters of cells with similar gene expression profiles.

Methods that rely on predefined markers (such as e.g. CellAssign) require manual curation and often extensive empirical evidence to select the panel of cell-type markers and determining parameters for the clustering algorithm. Methods that are based on reference data sets (such as e.g. CaSTLe) rely on a large number of genes to predict the cell types, resulting in models that have limited interpretability and are computationally intensive. Models trained at least partially on bulk RNA-seq data (such as e.g. SingleR) may suffer from limited generalizability due to the differences between the properties of bulk and single-cell data.

There remains an unmet need for methods and biomarkers that can be used to perform reliable and interpretable cell-type assignment using single cell transcriptomics data. The present invention addresses these and other needs.

### BRIEF DESCRIPTION OF THE INVENTION

Broadly, the present inventors leveraged the data generated from multi-modal sequencing platforms that simultaneously measure the levels of the mRNA and the proteins in individual cells, providing a direct link between the single-cell gene expression profiles and the cell types as identified based on validated surface protein markers. They developed a machine-learning-based feature selection workflow that probabilistically identifies a panel of cell-type predictive genes using the single-cell multi-modal data. The process does not rely on clustering analysis on the RNA-seq data or the empirically selected genes from the immunostaining marker panels. Applying the principle of parsimony to this process, the inventors identified a small panel of genes that achieves a high accuracy of immune cell-type assignment with high generalizability and interpretability.

Significantly, this small panel of genes was found to accurately classify cells in single cell transcriptomics datasets between common immune cell-types, independently of the classification algorithm used, suggesting high biological interpretability and generalizability. By contrast, existing approaches to assign cell types using single cell transcriptomics datasets did not perform as well, were significantly more computationally intensive and/or relied on gene panels that are not as interpretable and unlikely to generalize.

Accordingly, a first aspect provides a method of identifying a predictor set of genes for predicting the cell type of one or more cells. The method comprises:(a) obtaining a single cell gene expression profile comprising gene expression measurements for a set of genes, for a plurality of cells, and a single cell protein expression profile comprising protein expression measurements for two or more proteins, for the plurality of cells;(b) using the single cell protein expression profiles and an unsupervised learning method to assign a cell type class to at least some of the plurality of cells; and (d) applying a feature selection process to the single gene expression profiles to identify genes in the single cell gene expression profiles that are predictive of the cell type classes assigned in step (b), wherein the genes identified in step (d) form a predictor set of genes for predicting the cell type of one or more cells. The method optionally comprises (c) scaling and/or discretising the measurements for each gene in the single cell gene expression profiles.

The present inventors have discovered that matched single cell gene expression and protein expression data could be used to identify compact predictor sets of genes that can be used to accurately predict the cell type of one or more cells, using the above method.

Matched single cell gene expression and protein expression data can be obtained from combined single cell transcriptomics and proteomics protocols, and in particular from any multi-modal sequencing platform (e.g. 3′ feature barcoding from 10X Genomics, CITE-Seq, etc.) or from protocols that combine a proteomics detection step (e.g. FACS) and a transcriptomics detection set (e.g. scRNAseq). A multi-modal single cell sequencing platform as used herein refers to a platform that measures expression of one or more proteins and one or more genes through a single nucleic acid sequencing step. By contrast, a single cell platform or protocol that combines a proteomics detection step and a transcriptomics detection step measures expression of one or more proteins and one or more genes using separate detection steps (such as e.g. detection of fluorescent tags for the proteomic step followed by sequencing of the transcriptome of the cell), where the results of the separate detection steps for a single cell can be mapped to the cell.

Once the predictor set of genes has been identified, this can be used to accurately predict the cell type of one or more cell from any single cell gene expression data. For example, any single cell transcriptomics data set can be analysed using the predictor set of genes to identify the cell type of the cells from which the transcriptomics data set has been obtained.

Advantageously, this means that accurate cell type identification can be performed without the need to measure any protein markers, which are typically used as gold standard for cell type identification.

The claimed method does not rely on bulk RNA sequencing data at any point in the process. In particular, single cell gene expression profiles are only compared to each other in order to predict a cell type class. Without wishing to be bound by theory, this is believed to be advantageous because gene expression markers that have been found to be indicative of cell type in bulk RNA sequencing data may not be indicative of cell type at the level of single cells. For example, a gene expression marker that is expressed at high level by a small subset of cells in a particular cell type may be predictive of the cell type in a bulk RNA sequencing experiment while having low to no predictive value in a single cell RNA sequencing experiment.

Further, the claimed method also does not rely on manual (expert) input to define a predictor set of genes. Indeed, these are identified in an unbiased manner, directly from the gene expression data, using ground truth data for the cell type labels derived from single cell protein marker expression.

Advantageously, due to the use of an unsupervised learning method in assigning a cell type for use in feature selection, the method does not rely on manual input to identify groups of cells that are likely to be the same cell type. Instead, it uses single cell proteomics data to identify groups of cells that are phenotypically related (as indicated by similarity in their protein expression profiles) and hence likely to belong to the same cell type. Without wishing to be bound by theory, it is believed that such an approach more accurately captures heterogeneity and noise in protein expression within a cell type. For example, a small proportion of cells of one type may express a protein marker that is commonly believed to be associated with another cell type. Data driven identification of groups of cells is likely to enable the grouping of these cells with other cells of the same cell type, whereas labelling based purely on expression of a specific protein marker would associate these cells with the other cell type. This may result in a decreased ability to identify gene expression markers that are predictive of the respective cell types.

The single cell gene expression profile is preferably one that has been obtained using a high-throughput transcriptomics technology. For example, the single cell gene expression profile may comprise gene expression measurements for a set of genes comprises at least 100 genes, at least 500 genes, or at least 1000 genes. The single cell gene expression profile may be a substantially whole transcriptome gene expression profile. In some cases, the high-throughput transcriptomics technology is an untargeted transcriptomics technology, for example using next-generation sequencing. In other words, the single cell gene expression profile may have been obtained using a technology that aims to identify substantially all transcripts expressed by a cell. As the skilled person understands, not all transcripts that can theoretically be expressed from a cell's genome will be expressed in any particular condition, and technologies such as next-generation sequencing typically sample the transcriptome of a cell such that not all transcripts expressed by the cell may in fact be detected. Suitably, the single cell gene expression profile has been obtained through single cell RNA sequencing.

Preferably, the plurality of cells comprises at least 100 cells, at least 500 cells, or at least 1000 cells. Without wishing to be bound by theory, it is believed that increasing the number of cells in the plurality of cells may increase the richness of the data set used to identify predictor genes and as such may result in a more robust predictor set of genes.

Preferably, at least one of the proteins in the single cell protein expression profile is a known protein marker of cell type. In other words, at least one of the proteins in the single cell protein expression profile is preferably chosen based on prior knowledge of its expression being an indicator of cell type. For example, expression of the protein CD19 is commonly used as an indication of a cell being a B lymphocyte (where a cell that expresses CD19 may be considered likely to be a B lymphocyte). As another example, expression of the proteins CD3, CD4, CD8 and/or CD25 is commonly used as an indication of a cell being a T lymphocyte. Indeed, a cell that expresses CD3 may be considered likely to be a T lymphocyte. Further, subtypes of cells may be distinguishable for example on the basis of expression of CD4, CD8 and/or CD25. A cell that expresses CD3 and CD4 may be considered likely to be a CD4 T lymphocyte. A cell that expresses CD3 and CD8 (or CD3 but not CD4) may be considered likely to be a CD8 T lymphocyte. As another example, expression of the proteins CD56 and/or CD3 is commonly used as an indication of a cell being a natural killer cell. Indeed, a cell that expresses CD56 may be considered likely to be a natural killer cell, especially if the cell also does not express CD3. As another example, expression of the proteins CD14, CD15, CD16, CD4 and/or CD3 is commonly used as an indication of a cell being a monocyte. Indeed, a cell that expresses CD14 but does not express CD3, or expresses CD4 but not CD14 and CD3, or expresses CD15 and CD16 but not CD14 or CD3 may be considered likely to be a monocyte. As another example, expression of the protein CD34 is commonly used as an indication of a cell being a progenitor cell.

Indeed, a cell that expresses CD34 may be considered likely to be a progenitor cell. While the examples above primarily relate to immune cell types, as the skilled person understand, protein expression markers have been used for cell type identification for various cell types, and many cell types have commonly accepted protein markers that have been used e.g. in immunohistochemistry or FACS protocols for many years.

In some cases, the single cell protein expression profile comprises protein expression measurements for at least 3, at least 4, or at least 5 proteins. Without wishing to be bound by theory, it is believed that the protein expression profiles comprising measurements for increasing numbers of proteins will result in increasingly rich data sets for unsupervised learning, providing a more complex picture of the subpopulations of cells that may be present. This may increase the ability of the unsupervised learning method to identify biologically relevant subsets of cells. As a result, types of cells may be identified in step (b) with more confidence and/or subtypes of cells may be identifiable which may not have been distinguishable from each other using fewer proteins.

In embodiments, the unsupervised learning method is a clustering method. Any clustering method known in the art may be used. For example, the clustering method may be a linkage based clustering (e.g. hierarchical clustering), a centroid based clustering (e.g. k-means), a distribution-based clustering (e.g. Gaussian mixture models), a density-based clustering, a graph-based clustering (e.g. clique analysis), or an unsupervised neural network (e.g. a self-organising map).

In embodiments, using the single cell protein expression profiles and an unsupervised learning method to assign a cell type class to at least some of the plurality of cells comprises clustering the single cell protein expression profiles to identify at least a first group of cells and a second group of cells, and assigning a common cell type class to cells in at least one of the groups using the protein expression profiles of the cells in said group.

In embodiments, assigning a common cell type class to cells in at least one of the groups using the protein expression profiles of the cells in said group comprises defining one or more rules that apply to the measurements for one or more of the two or more proteins in the single cell protein expression profile. In embodiments, the one or more rules may apply to the measurements for one or more proteins that are known protein markers of cell types (i.e. protein markers known to be associated with a cell type).

For example, a group may be assigned a common cell type class if the proportion of cells in the group expressing one or more protein markers associated with the cell type is above a threshold. Alternatively, a group may be assigned a common cell type class if the average or median expression measurements for one or more protein markers associated with the cell type across cells in the group is above a threshold.

Without wishing to be bound by theory, it is believed that a population of cells even from a single cell type may not all express a particular known protein marker or combination of markers. As such, the use of a protein expression profile comprising measurements for at least two proteins increases the chances of identifying groups of cells that are similar to each other and as such are likely to be of the same cell type, using an unsupervised learning method. Once these groups of cells have been identified, expression of the known protein marker can be used to assign labels to the groups. This process is believed to reflect the biology of cell type populations more accurately than would be possible if all cells were assigned a cell type simply on the basis of whether they express or do not express a known protein marker.

In embodiments, clustering the single cell protein expression profiles comprises building a graph comprising nodes and edges, where nodes correspond to cells and edges link cells that have a similar protein expression profile, and identifying groups of cells as sets of interconnected nodes. Similarity may be measured using distances between protein expression profiles, correlations between expression profiles, and/or the Jaccard similarity coefficient.

In embodiments, the predictor set of genes comprises at most at most 100 genes, at most 90 genes, at most 80 genes, at most 70 genes, at most 60 genes, at most 50 genes, at most 40 genes, at most 30 genes, or at most 20 genes.

In embodiments, the predictor set of genes, when used as predictive features of a classification algorithm results in a classification algorithm that predicts cell types corresponding to the cell type classes of step (b) with an accuracy of at least 70%, at least 80% or at least 90%, using the single cell gene expression profiles obtained at step (a).

The present inventors have identified that the present method allowed to identify compact predictor sets of genes that could predict the cell type of a cell with high accuracy. Such compact predictors advantageously result in classifiers that have high computational efficiency. As a result, it is possible to predict the cell types of very high number of individual cells quicker than with methods of the prior arts, such as e.g. methods that use whole transcriptome data as predictive features.

In particular, the predictor set of genes preferably has a predictive accuracy of at least 70% for the training data. Accuracy for a training data set can be assessed for example using cross-validation. Accuracy of a classifier may be determined by calculating the AUROC. Preferably, the classification algorithm also has high predictive accuracy for one or more validation data sets, i.e. using single cell gene expression profiles other than those obtained at step (a).

In embodiments, the method further comprises normalising the single cell gene expression profiles relative to each other. Preferably, normalisation is performed prior to scaling and/or discretising, if used.

In embodiments, the method comprises scaling the measurements for each gene in the single cell gene expression profiles between 0 and 1. Scaling may comprise a linear mapping of the range of minimum to maximum expression for each gene to the [0,1] interval.

Scaling single cell gene expression data (in particular, scRNA-seq data) between 0 and 1 advantageously makes it relatively straightforward to define a common threshold that separates the cells in the first subset (which may be assumed to correspond to a zero peak) from the cells in the second subset, for all genes.

In embodiments, discretising the measurements for each gene in the single cell gene expression profiles comprises binarising the measurements. In other embodiments, discretising the measurements for each gene in the single gene expression profiles comprises assigning one of 3 or more discrete values to each measurement. For example, discretising the measurements for each gene may comprise defining 3 or more subranges of expression measurements for a gene and assigning one of 3 or more discrete values to each measurement depending on the subrange in which the measurement falls.

In embodiments, the method comprises binarising the measurements for each gene in the single cell gene expression profiles.

Binarisation of the data was found to lead to models that are more robust, i.e. models that generalise well between a training data set and a validation data set. In other words, without wishing to be bound by theory, it is believed that the binarisation helps to reduce the risk of overfitting the model to the training data.

In embodiments, binarising the measurements for each gene comprises assigning a first Boolean value to measurements below a threshold and a second Boolean value to measurements above a threshold. In embodiments, a single threshold may be used. In embodiments, two distinct thresholds may be used, and measurements between the thresholds may be considered as undetermined. In embodiments, a predetermined threshold or thresholds may be used for all genes. In embodiments, a threshold or thresholds may be chosen separately for each gene. In embodiments, a threshold or thresholds may be selected by investigating the distribution of expression values for a gene across cells. For example, a threshold may be chosen by fitting a mixture model to the distribution of expression values for a gene, and selecting a threshold as the value that optimally separates two distributions or groups of distributions. In embodiments where scaled data is used, a predetermined threshold between 0.01 and 0.1 may be used.

In embodiments, the method comprises log transforming the measurements for each protein in the single cell protein expression profile, prior to applying the unsupervised learning method to assign a cell type class to at least some of the plurality of cells.

The inventors advantageously found that log transformed single cell protein expression data often shows a bimodal distribution with a clear separation of cells that can be considered to be positive and negative for expression of a marker.

In embodiments, the method comprises binarising the measurements for each protein in the single cell protein expression profile.

In embodiments, binarising the measurements for each protein comprises assigning a first Boolean value to measurements below a threshold and a second Boolean value to measurements above a threshold. In embodiments, a single threshold may be used. In embodiments, two distinct thresholds may be used, and measurements between the thresholds may be considered as undetermined. In embodiments, a predetermined threshold or thresholds may be used for all proteins. In embodiments, a threshold or thresholds may be chosen separately for each protein. In embodiments, a threshold or thresholds may be selected by investigating the distribution of expression values for a protein across cells. For example, a threshold may be chosen by fitting a mixture model to the distribution of expression values for a protein, and selecting a threshold as the value that optimally separates two distributions or groups of distributions.

In embodiments, applying a feature selection process comprises training one or more classifiers to predict the cell type classes assigned in step (b) using the single gene expression profiles as input variables.

In embodiments, training one or more classifiers comprises training one or more boosted tree models.

In embodiments, wherein applying a feature selection process comprises training a plurality of classifiers to predict the cell type classes assigned in step (b) using the single gene expression profiles as input variables, and identifying genes in the single gene expression profiles that are predictive of the cell type classes assigned in step (b) comprises comparing the genes used by the plurality of classifiers in making a prediction and selecting those genes that are used by two or more of the plurality of classifiers.

In embodiments, obtaining a single cell gene expression profile and a single cell protein expression profile for a plurality of cells comprises obtaining a single cell gene expression profile and a single cell protein expression profile for a first plurality of cells, and for at least a further plurality of cells, wherein the expression profiles for the first and further plurality of cells were obtained through separate experiments, preferably wherein steps (b), (c) and/or (d) are performed independently for the first and further plurality of cells.

In embodiments, the feature selection process of step (d) is performed independently for the first and further plurality of cells, and genes in the single gene expression profiles that are predictive of the cell type classes assigned in step (b) are identified based on the combined outputs of the independent feature selection processes.

In embodiments, the first and one or more further plurality of cells have been obtained from at least two different types of samples, where the samples may differ by e.g. their tissue of origin, and/or wherein the expression profiles for the first and one or more further plurality of cells have been obtained using at least two different experimental protocols.

In embodiments, applying a feature selection process comprises training a plurality of classifiers to predict the cell type classes assigned in step (b) using the single gene expression profiles as input variables, and identifying genes in the single gene expression profiles that are predictive of the cell type classes assigned in step (b) comprises comparing the genes used by the plurality of classifiers in making a prediction and selecting those genes that are used by two or more of the plurality of classifiers.

In embodiments, obtaining a single cell gene expression profile and a single cell protein expression profile for a plurality of cells comprises obtaining a single cell gene expression profile and a single cell protein expression profile for a first plurality of cells, and for at least a further plurality of cells, wherein the expression profiles for the first and further plurality of cells were obtained through separate experiments.

In such embodiments, steps (b) and (c) (if used) may be performed independently for the first and further plurality of cells. Further, the feature selection process of step (d) may also be performed independently for the first and further plurality of cells, and genes in the single gene expression profiles that are predictive of the cell type classes assigned in step (b) may be identified based on the combined outputs of the independent feature selection processes.

Indeed, the inventors observed that some genes were expressed in a cell type in one data set but not in other data sets. In theory, such differences between data sets could be caused by various factors such as biological conditions, tissue types, batch effects, or protocols. These differences make it more difficult to identify which genes are genuine cell type markers by looking at a single data set. Further, the inventors identified that a conservative (parsimonious) approach to identify the genes that are predictive to the cell types across multiple data sets based on the combined outputs of separate feature selection processes performed even better than merging multiple data sets and performing the feature selection directly on such a merged data set. In particular, the inventors identified that if a gene was consistently selected as a predictive marker in all the subsets of cells from multiple training data sets, it is likely to be a marker that would be indicative of the cell type regardless of the conditions (or any other parameters of the data set that may influence the results).

In embodiments, identifying genes in the single gene expression profiles that are predictive of the cell type classes assigned in step (b) based on the combined outputs of the independent feature selection processes comprises identifying genes as predictive if they are identified as predictive in each of the independent feature selection processes.

In embodiments, identifying genes in the single gene expression profiles that are predictive of the cell type classes assigned in step (b) based on the combined outputs of the independent feature selection processes comprises identifying genes as predictive if they are identified as predictive in two or more of the independent feature selection processes.

In embodiments, identifying genes in the single gene expression profiles that are predictive of the cell type classes assigned in step (b) based on the combined outputs of the independent feature selection processes comprises identifying genes as predictive if they are identified as predictive in more than half of the independent feature selection processes.

In embodiments, the first and one or more further plurality of cells may have been obtained from at least two different types of samples, where the samples may differ by e.g. their tissue of origin.

In embodiments, the expression profiles for the first and one or more further plurality of cells may have been obtained using at least two different experimental protocols.

Without wishing to be bound by theory, it is believed that the use of data sets that vary in terms of experimental platforms and/or biological sample of origin may result in a more robust set of predictor genes.

In embodiments, steps (b) to (d) are computer implemented. Indeed, the size of matched single cell gene expression and protein expression data sets usable for the purpose of this method, in terms of the number of cells and/or the size of at least the single cell gene expression profiles is such that steps (b) to (d) are far beyond the capability of mental investigation.

In some cases, step (a) comprises processing one or more samples of cells or tissues using a combined single cell transcriptomics and proteomics protocol. Alternatively, step (a) may be computer-implemented and comprise receiving a previously acquired single cell gene expression profile and a previously acquired single cell protein expression profile for the plurality of cells.

According to a second aspect, there is provided a method for predicting the cell type of one or more cells, the method comprising:(i) obtaining a predictor set of genes, wherein the predictor set of genes has been identified using the method of any embodiment of the first aspect; (ii) obtaining a single cell gene expression profile for each of the one or more cells comprising gene expression measurements for a set of genes comprising at least 1, 2, 3, 4, 5, 6, 7 or more (such as all of) the predictor set of genes; and (iii) making a prediction of the cell type of the one or more cells based at least in part on the gene expression profile for the predictor set of genes.

In embodiments, obtaining a predictor set of genes comprises identifying a predictor set of genes using the method of any embodiment of the first aspect. In embodiments, obtaining a predictor set of genes comprises a computing device receiving a predictor set of genes or retrieving the predictor set of genes from a memory associated with the computing device.

In embodiments, obtaining a single cell gene expression profile for each of the one or more cells comprising gene expression measurements for a set of genes comprising at least 1, 2, 3, 4, 5, 6, 7 or more (such as all of) the predictor set of genes comprising performing single cell RNA sequencing or single cell RT-qPCR, or receiving the results of a previously performed single cell RNA sequencing or single cell RT-qPCR experiment.

Embodiments of this aspect may include any of the features described herein in relation to method for predicting the cell type of one or more cells. In particular, any of the features described in relation to the seventh aspect below are specifically envisaged in combination with the present aspect.

According to a third aspect, there is provided a system comprising: at least one processor; and at least one non-transitory computer readable medium containing instructions that, when executed by the at least one processor, cause the at least one processor to perform the method of any embodiment of the first or second aspect.

In embodiments, obtaining a single cell gene expression profile and a single cell protein expression profile for a plurality of cells, comprises processing one or more samples of cells or tissues using a combined single cell transcriptomics and proteomics protocol. For example, the combined single cell transcriptomics and proteomics protocol may be chosen from 3′ feature barcoding from 10X Genomics, CITE-Seq, combined FACS and scRNAseq (e.g. using drop-seq or GemCode), etc. In embodiments, obtaining a single cell gene expression profile and a single cell protein expression profile for a plurality of cells comprises processing one or more samples in vitro.

According to a fourth aspect, there is provided a computer implemented method of identifying a predictor set of genes for predicting the cell type of one or more cells, the method comprising:(a) receiving a single cell gene expression profile comprising gene expression measurements for a set of genes, for a plurality of cells, and a single cell protein expression profile comprising protein expression measurements for two or more proteins, for the plurality of cells; (b) using the single cell protein expression profiles and an unsupervised learning method to assign a cell type class to at least some of the plurality of cells; (c) optionally scaling and/or discretising the measurements for each gene in the single cell gene expression profiles; (d) applying a feature selection process to the single gene expression profiles to identify genes in the single gene expression profiles that are predictive of the cell type classes assigned in step (b), wherein the genes identified in step (d) form a predictor set of genes for predicting the cell type of one or more cells.

According to a fifth aspect, there is provided a system for identifying a predictor set of genes for predicting the cell type of one or more cells, the system comprising: at least one processor; and at least one non-transitory computer readable medium containing instructions that, when executed by the at least one processor, cause the at least one processor to perform the method of the preceding aspect of the invention.

In a sixth aspect, the present invention provides a non-transitory computer readable medium for identifying a predictor set of genes for predicting the cell type of one or more cells, comprising instructions that, when executed by at least one processor, cause the at least one processor to perform the method of the preceding aspect of the invention.

According to a seventh aspect, there is provided a method for predicting the cell type of one or more cells, the method comprising: a) obtaining a single cell gene expression profile for each of the one or more cells comprising gene expression measurements for a set of genes comprising at least 2, 3, 4, 5, 6, 7 or more (such as all of) the following genes: CST3, CD8B, IL7R, KLRF1, MS4A1, CD8A, TRBC2, CD3D, GZMK, NKG7, CD79A, TYROBP, IL32, CD3E, CTSW, GZMB, CD4, CD69, and TRAC; and b) making a prediction of the cell type of the one or more cells based at least in part on the gene expression profile for a predictor set of genes comprising at least 2, 3, 4, 5, 6, 7 or more of (such as all of) the following genes: CST3, CD8B, IL7R, KLRF1, MS4A1, CD8A, TRBC2, CD3D, GZMK, NKG7, CD79A, TYROBP, IL32, CD3E, CTSW, GZMB, CD4, CD69, and TRAC.

Where the single cell gene expression profile is obtained using a whole transcriptome and/or an untargeted technology (such as e.g. single cell RNA sequencing), the predictor set of genes is a subset of the genes represented in the single cell gene expression profile. In other words, the prediction is not based on a whole transcriptome expression profile.

In embodiments, the predictor set of genes comprises at least 4 of the following genes: CST3, CD8B, IL7R, KLRF1, MS4A1, CD8A, TRBC2, CD3D, GZMK, NKG7, CD79A, TYROBP, IL32, CD3E, CTSW, GZMB, CD4, CD69, and TRAC.

In embodiments, the predictor set of genes comprises: at least one gene selected from a first subset comprising: KLRF1, NKG7, CTSW, TYROBP, and GZMB; at least one gene selected from a second subset comprising: CD79A, and MS4A1; at least one gene selected from a third subset comprising: CST3, CD4 and TYROBP; and at least one gene selected from a fourth subset comprising: CD3D, CD3E, TRAC, TRBC2, IL32, IL7R, and CD69.

The present inventors have identified that such sets of genes are robust cell type predictors from single cell gene expression data. In particular, the above selection of predictor genes was found to reliably distinguish between at least B cells, monocytes and T cells.

In embodiments, the predictor set of genes further comprises at least one gene selected from a fifth subset comprising: CD8A, CD8B, CSTW, NKG7, GZMK, GZMB, and CD4.

In embodiments, the set of genes comprises at least 5 genes and the prediction is made based at least in part on the gene expression profile for at least one gene from each of the first to fifth subsets, wherein at least one gene selected from each subset is different from the at least one gene selected from the other subsets.

In embodiments, the predictor set of genes includes at most 100 genes, at most 90 genes, at most 80 genes, at most 70 genes, at most 60 genes, at most 50 genes, at most 40 genes, at most 30 genes, or at most 20 genes, optionally wherein the predictor set of genes does not comprise more than 5, more than 10, more than 15 or more than 20 genes that are not selected from: CST3, CD8B, IL7R, KLRF1, MS4A1, CD8A, TRBC2, CD3D, GZMK, NKG7, CD79A, TYROBP, IL32, CD3E, CTSW, GZMB, CD4, CD69, and TRAC.

In embodiments, making a prediction of the cell type of the one or more cells is performed based solely on the gene expression profile for the predictor set of genes.

The present inventors have identified that such sets of genes are robust cell type predictors from single cell gene expression data, and in particular can reliably distinguish between at least B cells, monocytes, natural killer (NK) cells, CD8+ T cells and non-CD8+ T cells.

Making a prediction of the cell type of the one or more cells based at least in part on the gene expression profile for the predictor set of genes may comprise making a prediction of the cell type of the one or more cells based on the gene expression profile for a predictor set of at most 100 genes, at most 90 genes, at most 80 genes, at most 70 genes, at most 60 genes, at most 50 genes, at most 40 genes, at most 30 genes, or at most 20 genes. In some embodiments, the predictor set of genes does not comprise more than 5, more than 10, more than 15 or more than 20 genes that are not selected from: CST3, CD8B, IL7R, KLRF1, MS4A1, CD8A, TRBC2, CD3D, GZMK, NKG7, CD79A, TYROBP, IL32, CD3E, CTSW, GZMB, CD4, CD69, and TRAC.

Advantageously, the set of genes as claimed may be sufficient to reliably predict the cell type of one or more cells such that an accurate prediction can be obtained using succinct panels comprising these genes.

In embodiments, making a prediction of the cell type of the one or more cells based at least in part on the gene expression profile for the predictor set of genes may comprise making a prediction of the cell type of the one or more cells based solely on the gene expression profile for the predictor set of genes. In some such embodiments, the predictor set of genes comprises at 100 genes, at most 90 genes, at most 80 genes, at most 70 genes, at most 60 genes, at most 50 genes, at most 40 genes, at most 30 genes, or at most 20 genes. In some such embodiments, the predictor set of genes does not comprise more than 5, more than 10, more than 15 or more than 20 genes that are not selected from: CST3, CD8B, IL7R, KLRF1, MS4A1, CD8A, TRBC2, CD3D, GZMK, NKG7, CD79A, TYROBP, IL32, CD3E, CTSW, GZMB, CD4, CD69, and TRAC.

In embodiments, the predictor set of genes comprises or consists of CST3, CD8B, IL7R, KLRF1 and MS4A1.In embodiments, wherein the predictor set of genes comprises CST3, CD8B or CD8B, IL7R and/or TRBC2 and/or CD3D, KLRF1 and MS4A1. In embodiments, the predictor set of genes comprises CST3, CD8B, IL7R, KLRF1 and MS4A1. In embodiments, the predictor set of genes consists of CST3, CD8B, IL7R, KLRF1 and MS4A1.

The present inventors have discovered that CST3, CD8B, IL7R, KLRF1 and MS4A1 are each able to identify a particular cell type with high confidence, together forming a set that is sufficient to discriminate between the five cell types that each of these gene identifies.

In embodiments, the predictor set of genes comprises CST3, CD8B or CD8B, IL7R and/or TRBC2 and/or CD3D, KLRF1 and MS4A1. In embodiments, the predictor set of genes consists of CST3, CD8B or CD8B, IL7R and/or TRBC2 and/or CD3D, KLRF1 and MS4A1.

In embodiments, the predictor set of genes comprises CST3, CD8B, IL7R, KLRF1, MS4A1, CD8A, TRBC2, CD3D, GZMK, NKG7, CD79A, TYROBP, IL32, CD3E, CTSW, GZMB, CD4, CD69 and TRAC. In embodiments, the predictor set of genes consists of CST3, CD8B, IL7R, KLRF1, MS4A1, CD8A, TRBC2, CD3D, GZMK, NKG7, CD79A, TYROBP, IL32, CD3E, CTSW, GZMB, CD4, CD69 and TRAC.

In embodiments, the one or more cells are mammalian cells, preferably human cells. In embodiments, the one or more cells are healthy or cancerous immune cells.

In embodiments, the one or more cells are immune cells, such as a lymphoid tissue cells or a white blood cells. For example, the one or more cells may (each) be a PBMC, a CBMC, a spleen mononuclear cell (SMC), a bone marrow mononuclear cell (BMMC), a mucosa-associated lymphoid tissue (MALT) cell, a thymus cell, a lymph node cell, or a tonsil cell.

In embodiments, the one or more cells are each selected from: a healthy immune cell and a cancerous immune cell. A cancerous immune cell may be a lymphoma cell, a leukemia cell or a myeloma cell.

In embodiments, the one or more cells are white blood cells, such as mononuclear blood cells. For example, a cell may be a peripheral blood mononuclear cell (PBMC) or a cord blood mononuclear cell (CBMC).

Making a prediction of the cell type of the one or more cells based at least in part on the gene expression profile for the predictor set of genes may comprise classifying each of the one or more cells between two or more cell type classes.

In embodiments, the two or more cell type classes comprise at least a first class, a second class, a third class, a fourth class and a fifth class, wherein cells classified in the first class are predicted to be B cells, cells classified in the second class are predicted to be CD8+ T cells, cells classified in the third class are predicted to be monocytes, cells classified in the fourth class are predicted to be NK cells, and cells classified in the fifth class are predicted to be non CD8+ T cells.

In embodiments, the two or more cell type classes comprise a first class and a second class, wherein cells classified in the first class are predicted to be monocytes and cells classified in the second class are predicted to not be monocytes. In such embodiments, the predictor set of genes preferably comprises two or more genes selected from the third subset.

In embodiments, the two or more cell type classes comprise a first class and a second class, wherein cells classified in the first class are predicted to be B lymphocytes and cells classified in the second class are predicted to not be B lymphocytes. In such embodiments, the predictor set of genes preferably comprises two or more genes selected from the second subset.

In embodiments, the two or more cell type classes comprise a first class and a second class, wherein cells classified in the first class are predicted to be T cells and cells classified in the second class are predicted to not be T cells. In such embodiments, the predictor set of genes preferably comprises two or more genes selected from the first, fourth or fifth subset.

In embodiments, the two or more cell type classes comprise a first class and a second class, wherein cells classified in the first class are predicted to be natural killer cells and cells classified in the second class are predicted to not be natural killer cells. In such embodiments, the predictor set of genes preferably comprises one or more genes selected from the first subset.

In embodiments, the two or more cell type classes comprise a first class and a second class, wherein cells classified in the first class are predicted to be CD8+ T cells and cells classified in the second class are predicted to not be CD8+ T cells. In such embodiments, the predictor set of genes preferably comprises one or more genes selected from the fifth subset.

In embodiments, the two or more cell type classes comprise a first class and a second class, wherein cells classified in the first class are predicted to be CD8− T cells and cells classified in the second class are predicted to not be CD8− T cells. In such embodiments, the predictor set of genes preferably comprises one or more genes selected from the fourth subset.

In embodiments, the two or more cell type classes comprise a first class and a second class, wherein cells classified in the first class are predicted to be CD4+ T cells and cells classified in the second class are predicted to not be CD4+ T cells. In such embodiments, the predictor set of genes preferably comprises one or more genes selected from the fourth subset.

In embodiments, the two or more cell type classes comprise at least a first class, a second class and a third class, wherein cells classified in the first class are predicted to be B cells, cells classified in the second class are predicted to be T cells, and cells classified in the third class are predicted to be monocytes.

In embodiments, the two or more cell type classes comprise at least a first class, a second class, a third class and a fourth class, wherein cells classified in the first class are predicted to be B cells, cells classified in the second class are predicted to be T cells, cells classified in the third class are predicted to be monocytes, and cells classified in the fourth class are predicted to be NK cells.

In embodiments, the two or more cell type classes comprise at least a first class, a second class, a third class, a fourth class and a fifth class, wherein cells classified in the first class are predicted to be B cells, cells classified in the second class are predicted to be CD8+ T cells, cells classified in the third class are predicted to be monocytes, cells classified in the fourth class are predicted to be NK cells, and cells classified in the fifth class are predicted to be non CD8+ T cells.

In some cases making a prediction of the cell type based on the gene expression profile for a predictor set of genes comprises applying a computational classifier, such as a machine learning model, to said gene expression profile, wherein said computational classifier is adapted to assign an unknown gene expression profile to a cell type class based on a training set of sample gene expression profiles of said predictor set of genes obtained from training samples of known cell type.

The computational classifier may optionally have been subjected to multiple rounds of training in order to tune model parameters and/or optimise performance of the computational classifier on the training samples.

In some cases the computational classifier comprises a machine learning model. In particular, the classifier may comprise a support vector machine (SVM), a decision tree (or ensemble of decision trees) or a logistic regression classifier. In some such embodiments, the classifier comprises an ensemble of decision trees trained using gradient boosting.

In embodiments, obtaining a single cell gene expression profile for each of the one or more cells comprises performing single cell RNA sequencing. In embodiments, obtaining a single cell gene expression profile for each of the one or more cells comprises analysing the results of a single cell RNA sequencing experiment.

In embodiments, obtaining a single cell gene expression profile for each of the one or more cells comprises performing single cell RT-qPCR targeting a set of genes comprising at least 4, 5, 6, 7 or more (such as all of) the following genes: CST3, CD8B, IL7R, KLRF1, MS4A1, CD8A, TRBC2, CD3D, GZMK, NKG7, CD79A, TYROBP, IL32, CD3E, CTSW, GZMB, CD4, CD69, and TRAC.

In embodiments, making a prediction of the cell type of the one or more cells based at least in part on the gene expression profile for the predictor set of genes comprises discretising the gene expression profile for the predictor set of genes. For example, discretising the gene expression profile for the predictor set of genes may comprise identifying gene expression measurements values that correspond to cells that are positive for expression of a gene in the predictor set of gene and gene expression measurement values that correspond to cells that are negative for expression of the gene in the predictor set of genes.

Making a prediction of the cell type of the one or more cells based at least in part on the gene expression profile for the predictor set of genes may comprise predicting that the cell is a monocyte if its gene expression profile indicates that the cell is positive for expression of one or both of CST3, CD4 and TYROBP.

Making a prediction of the cell type of the one or more cells based at least in part on the gene expression profile for the predictor set of genes may comprise predicting that the cell is a B lymphocyte if its gene expression profile indicates that the cell is positive for expression of one or both of CD79A, and MS4A1.

Making a prediction of the cell type of the one or more cells based at least in part on the gene expression profile for the predictor set of genes may comprise predicting that the cell is a T lymphocyte if its gene expression profile indicates that the cell is positive for expression of one or more of KLRF1, NKG7, CTSW, GZMB, CD3D, CD3E, TRAC, TRBC2, IL32, IL7R, CD69, CD8A, CD8B, CSTW, GZMK, and CD4. Making a prediction of the cell type of the one or more cells based at least in part on the gene expression profile for the predictor set of genes may comprise predicting that the cell is a T lymphocyte if its gene expression profile indicates that the cell is positive for expression of one or more of CD3E, CD3D, TRAC, IL32, IL7R, and CD69.

Making a prediction of the cell type of the one or more cells based at least in part on the gene expression profile for the predictor set of genes may comprise predicting that the cell is a NK cell if its gene expression profile indicates that the cell is positive for expression of one or more of KLRF1, NKG7, CTSW, TYROBP and GZMB.

Making a prediction of the cell type of the one or more cells based at least in part on the gene expression profile for the predictor set of genes may comprise predicting that the cell is a CD8+ T lymphocyte if its gene expression profile indicates that the cell is positive for expression of one or more of CD8A, CD8B, CSTW, NKG7, GZMK, GZMB, and CD4. Making a prediction of the cell type of the one or more cells based at least in part on the gene expression profile for the predictor set of genes may comprise predicting that the cell is a CD8+ T lymphocyte if its gene expression profile indicates that the cell is positive for expression of one or more of CD8A, CD8B, CSTW, NKG7, GZMK, GZMB, and CD4 and one or more of CD3E, CD3D, TRAC, IL32, IL7R, and CD69.

Making a prediction of the cell type of the one or more cells based at least in part on the gene expression profile for the predictor set of genes may comprise predicting that the cell is a non-CD8+ T lymphocyte if its gene expression profile indicates that the cell is negative for expression of one or more of CD8A, CD8B, CSTW, NKG7, GZMK, GZMB, and CD4 and one or more of CD3E, CD3D, TRAC, IL32, IL7R, and CD69.

In some cases in accordance with the previous aspect of the present invention, making a prediction comprises comparing the gene expression profile for the predictor set of genes for one or more cells with at least one reference gene expression profile for said predictor set of genes, wherein said at least one reference gene expression profile comprises:
(i) a B lymphocyte reference gene expression profile, optionally derived from at least one B lymphocyte cell or cell population; and/or
(ii) a NK cell reference gene expression profile, optionally derived from at least one NK cell or cell population; and/or
(iii) a monocyte reference gene expression profile, optionally derived from at least one monocyte cell or cell population; and/or
(iv) a CD8+ T lymphocyte reference gene expression profile, optionally derived from at least one CD8+ T lymphocyte cell or cell population; and/or
(v) a non CD8+ T lymphocyte reference gene expression profile, optionally derived from at least one non CD8+ T lymphocyte 20 cell or cell population.

Preferably, the gene expression profile for a cell is compared with two or more, preferably all of (i) to (v). In particular, the gene expression profile for a cell may be compared with each of the one or more reference profiles and an assessment of best fit may be used to classify the cell as being of a particular cell type. In some cases, the gene expression profile for a cell is discretised (such as e.g. binary) and compared with one or more discretised (such as e.g. binary) reference gene expression profile(s).

In embodiments, the reference gene expression profiles have been pre-determined and are obtained by retrieval from a volatile or non-volatile computer memory or data store.

In embodiments, the gene expression profile for a cell is compared with each reference gene expression profile for closeness of fit using K-means clustering, model based clustering, non-negative matrix factorization, variants of factor analysis or principal component analysis.

In embodiments, making a prediction of the cell type of the one or more cells based at least in part on the gene expression profile for the predictor set of genes comprises normalising the gene expression profile.

For example, the measured expression level of each gene may be normalised relative to the expression level of one or more housekeeping genes. The gene expression profile may instead or in addition be normalised relative to the gene expression profile of other cells in a population (i.e. relative to other gene expression profiles in a gene expression data set).

For example, the gene expression profile may be normalised to reflect differences in single cell sequencing library sizes in a single cell RNA sequencing data set. In embodiments, making a prediction of the cell type of the one or more cells based at least in part on the gene expression profile for the predictor set of genes comprises scaling the gene expression profile. In particular, scaling the gene expression profile may include scaling the measured gene expression levels for each gene between 0 and 1 across a population of gene expression profiles.

In embodiments, making a prediction of the cell type of the one or more cells based at least in part on the gene expression profile for the predictor set of genes comprises discretising the (optionally normalised and/or scaled) gene expression profile. For example, discretising the gene expression profile may comprise assigning a first value (e.g. positive/1) if the (optionally normalised and/or scaled) gene expression measurement for a gene is above a threshold, and a second value (e.g. negative/0) if the gene expression measurement for a gene is below a threshold (which may be the same or a different threshold).

The prediction methods described herein are preferably computer implemented. Indeed, the scale of a typical single cell gene expression data set is typically such that analysis cannot practically be performed mentally.

According to a further aspect, there is provided a single cell qRT-PCR kit comprising primers targeting at least 4, 5, 6, 7 or more (such as all of) the following genes: CST3, CD8B, IL7R, KLRF1, MS4A1, CD8A, TRBC2, CD3D, GZMK, NKG7, CD79A, TYROBP, IL32, CD3E, CTSW, GZMB, CD4, CD69, and TRAC. Preferably, at least one gene is selected from a first subset comprising: KLRF1, NKG7, CTSW, and GZMB; at least one gene is selected from a second subset comprising: CD79A, and MS4A1; at least one gene is selected from a third subset comprising: CST3, and TYROBP; at least one gene is selected from a fourth subset comprising: CD3D, CD3E, TRAC, TRBC2, IL32, IL7R, and CD69. In embodiments, at least one gene is selected from a fifth subset comprising: CD8A, CD8B, CSTW, NKG7, GZMK, GZMB, and CD4.

In embodiments, the single cell qRT-PCR kit comprises primers targeting at any combination described herein (such as e.g. in relation to the seventh aspect) of the following genes: CST3, CD8B, IL7R, KLRF1, MS4A1, CD8A, TRBC2, CD3D, GZMK, NKG7, CD79A, TYROBP, IL32, CD3E, CTSW, GZMB, CD4, CD69, and TRAC.

According to a further aspect, there is a method of providing a single cell qRT-PCR kit, the method comprising identifying a predictor set of genes for predicting the cell type of one or more cells as described in relation to the first aspect, and designing a single cell qRT-PCR kit comprising primers targeting one or more (such as all of) the predictor set of genes.

According to a further aspect, there is provided a computer implemented method for predicting the cell type of one or more cells, the method comprising:
a) obtaining a single cell gene expression profile for each of the one or more cells comprising gene expression measurements for a set of genes comprising at least 4, 5, 6, 7 or more (such as all of) the following genes: CST3, CD8B, IL7R, KLRF1, MS4A1, CD8A, TRBC2, CD3D, GZMK, NKG7, CD79A, TYROBP, IL32, CD3E, CTSW, GZMB, CD4, CD69, and TRAC;
b) (i) optionally, normalising and/or scaling the single cell gene expression profile(s); (ii)comparing the single cell gene expression profile(s) for a predictor set of genes to two or more reference gene expression profiles as described herein;
c) classifying the single cell gene expression profile(s) as belonging to the group having the reference gene expression profile to which it is most closely matched; and
d) providing a prediction of cell type based on the classification made in step c).

In embodiments, the single cell gene expression profile(s) is/are compared with each reference gene expression profile for closeness of fit using K-means clustering, model based clustering, non-negative matrix factorization, variants of factor analysis or principal component analysis.

According to a further aspect, there is provided a computer implemented method for predicting the cell type of one or more cells, the method comprising: a) obtaining a single cell gene expression profile for each of the one or more cells comprising gene expression measurements for a set of genes comprising at least 4, 5, 6, 7 or more (such as all of) the following genes: CST3, CD8B, IL7R, KLRF1, MS4A1, CD8A, TRBC2, CD3D, GZMK, NKG7, CD79A, TYROBP, IL32, CD3E, CTSW, GZMB, CD4, CD69, and TRAC; b) optionally, normalising and/or scaling the single cell gene expression profile(s); c) classifying the single cell gene expression profile(s) as belonging to one of a plurality of cell type classes using a classifier that has been trained using gene expression profiles from cells with known cell type, for a predictor set of genes as described in relation to the seventh aspect; and d) providing a prediction of cell type based on the classification made in step c).

In embodiments, the classifier is a SVM classifier, a decision tree classifier, or a logistic regression classifier.

According to a further aspect, there is provided a computer implemented method for classifying a cell from its gene expression profile, the method comprising:
a) obtaining a single cell gene expression profile for each of the one or more cells comprising gene expression measurements for a set of genes comprising at least 4, 5, 6, 7 or more (such as all of) the following genes: CST3, CD8B, IL7R, KLRF1, MS4A1, CD8A, TRBC2, CD3D, GZMK, NKG7, CD79A, TYROBP, IL32, CD3E, CTSW, GZMB, CD4, CD69, and TRAC; and
b) optionally, normalising and/or scaling the single cell gene expression profile(s);
c) classifying the single cell gene expression profile(s) as belonging to one of a plurality of cell type classes using a classifier that has been trained using gene expression profiles from cells with known cell type, for a predictor set of genes as described in relation to the seventh aspect; and
d) providing a prediction of cell type based on the classification made in step c).

In embodiments, the classifier is a SVM classifier, a decision tree classifier, or a logistic regression classifier.

According to a further aspect, there is provided a system for classifying a cell from its gene expression profile, the system comprising:
at least one processor; and
at least one non-transitory computer readable medium containing instructions that, when executed by the at least one processor, cause the at least one processor to perform operations comprising:(a) receiving gene expression data representing gene expression measurements for a set of genes comprising at least 4, 5, 6, 7 or more (such as all of) the following genes: CST3, CD8B, IL7R, KLRF1, MS4A1, CD8A, TRBC2, CD3D, GZMK, NKG7, CD79A, TYROBP, IL32, CD3E, CTSW, GZMB, CD4, CD69, and TRAC, forming a gene expression profile for the cell;(b) optionally, normalising and/or scaling the single cell gene expression profile; (c) receiving reference gene expression data comprising single cell gene expression profiles for a plurality of cells with known cell types; (d) classifying the single cell gene expression profile as belonging to one of a plurality of cell type classes using a classifier that has been trained using the gene expression profiles in the reference gene expression data, for a predictor set of genes for a predictor set of genes as described herein, such as in relation to the seventh aspect; and (d) providing a prediction of cell type based on the classification made in step (d).

In some embodiments, the system is for use in the method of the seventh aspect of the invention.

According to a further aspect, there is provided a system for predicting a cell type for a cell, the system comprising: at least one processor; and at least one non-transitory computer readable medium containing instructions that, when executed by the at least one processor, cause the at least one processor to perform the method of the seventh aspect of the invention.

In a further aspect, the present invention provides a non-transitory computer readable medium for predicting a cell type for a cell, comprising instructions that, when executed by at least one processor, cause the at least one processor to perform the method of the seventh aspect of the invention.

In embodiments of any aspect, a cell of know cell type is a cell that has been assigned a cell type based at least in part on measurements of expression of one or more proteins for the cell.

Embodiments of the present invention will now be described by way of example and not limitation with reference to the accompanying figures. However various further aspects and embodiments of the present invention will be apparent to those skilled in the art in view of the present disclosure.

The present invention includes the combination of the aspects and preferred features described except where such a combination is clearly impermissible or is stated to be expressly avoided. These and further aspects and embodiments of the invention are described in further detail below and with reference to the accompanying examples and figures.

### BRIEF DESCRIPTION OF THE FIGURES

FIG. 1 is a flowchart illustrating a method of identifying a predictor set of genes for cell type assignment from single cell gene expression data as described herein.

FIG. 2 shows a bar plot of the importance of selected gene expression features in increasing the accuracy of the prediction of cell types from single cell transcriptomic data. For each gene, the plot shows the average Gain associated with adding splits based on the gene, to an ensemble of classifier trees that are being trained. The Gain quantifies the difference between the scores for the new leaves associated with a split and the score for the original leaf before the split, where the scores for leaves captures classification accuracy using a logistic loss function.

FIG. 3 shows a bar plot of the area under (AU) the ROC (receiver operator curve) for univariate binary classifiers using each of a set of selected genes as described herein. Each bar represents the AUROC for the prediction of whether a cell belongs to a particular cell type based on the level of expression of a single selected gene, in a specific data set.

FIG. 4 shows a comparison of multiple cell-type assignment models in terms of (multi-class) classification accuracy (A) and computational time to obtain a prediction (B), for each of the validation datasets. AMASC−XGBoost=boosted tree classifier using AMASC gene panel; AMASC−logistic=logistic regression classifier using AMASC gene panel; AMASC−SVM=support vector machine classifier using AMASC gene panel; SingleR(HPCA)=SingleR model using Human Primary Cell Atlas (HPCA) reference dataset; SingleR(Encode)=SingleR model using Encode reference dataset; CellAssign(TME)=CellAssign model using default panel; CellAssign(TME+AMASC)=CellAssign model using default panel extended to include AMASC gene panel. All predictions were obtained using the same processor (Intel® Xeon® E5-2680 v3).

FIG. 5 shows the UMAP clustering for a single cell protein marker data set from peripheral blood mononuclear cells (PBMC1OKV3). The protein markers used are CD3, CD8, CD4, CD14, CD19, and CD56. Cell populations were manually labelled based on expression of the markers (mono=monocytes, CD4 T=CD4+ T cells, CD8 T=CD8+ T cells, B=B lymphocytes, NK=natural killer cells).

FIGS. 6A and 6B show histograms of the raw (A) and log-transformed (B) CD3 protein expression data in the PBMC10KV3 data set. The raw data is measured in UMI (unique molecular index) counts (FIG. 6A, x axis).

### DETAILED DESCRIPTION OF THE INVENTION

In describing the present invention, the following terms will be employed, and are intended to be defined as indicated below.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

A “sample” as used herein may be a cell or tissue sample (e.g. a biopsy), a biological fluid, an extract (e.g. a protein or DNA extract obtained from the subject), from which single cells can be obtained for single cell gene expression analysis. In particular, the sample may be a blood sample, a purified blood sample such as a PBMC or CBMC sample, or a lymphoid tissue sample. The sample may be one which has been freshly obtained from a subject or may be one which has been processed and/or stored prior to making a determination (e.g. frozen, fixed or subjected to one or more purification, enrichment or extractions steps). Alternatively, the sample may be a cell or tissue culture sample. As such, a “cell” or “single cell” as described herein may refer to any type of cell, whether from a biological sample obtained from a subject, or from a sample obtained from e.g. a cell line. The cell is suitably a mammalian cell, preferably a human cell. The cell may further be a healthy cell or a diseased cell, such as e.g. a cancerous cell. For example, the cell may be a lymphoma cell.

Embodiments of the present disclosure relate to the identification of the cell type of immune cells. In such embodiments, a cell may be an immune cell, such as a lymphoid tissue cell or a white blood cell. For example, the cell may be a peripheral blood mononuclear cell (PBMC), a cord blood mononuclear cell (CBMC), a spleen mononuclear cell (SMC), a bone marrow mononuclear cell (BMMC), a mucosa-associated lymphoid tissue (MALT) cell, a thymus cell, a lymph node cell, or a tonsil cell.

### Cell Types

References to cell types refer to phenotypically and/or functionally distinct cell forms within an organism. Within the context of the present disclosure, a cell type refers to any class of cell that can be distinguished on the basis of expression of one or more protein markers. Embodiments of the present disclosure relate to the identification of the cell type of immune cells. Immune cells are commonly classified into phenotypically and/or functionally distinct classes including natural killer (NK) cells, B cells, monocytes, cytotoxic T cells (also referred to as CD8+ T cells), helper T cells (also referred to as CD4+ T cells), regulatory T cells (CD4+, CD25+ T cells), effector T cells, etc. Multiple subclassifications also exists such as e.g. naive CD8+ T cells, naive helper T cells, activated T cells, etc. Within the context of the present disclosure, one or more immune cell types may be distinguished on the basis of expression of one or more protein markers including: CD3 (T lymphocytes), CD4 (non-CD8+ T lymphocytes), CD8 (CD8+ T lymphocytes), CD19+ (B lymphocytes), CD56 (Natural killer cells), and CD14 (monocytes).

### Gene Expression

Reference to determining the expression level of a gene refers to determination of the expression level of an expression product of the gene. Within the context of the present invention, unless indicated otherwise, references to gene expression levels refer to gene expression determined at the nucleic acid level (i.e. at the transcript level). As such, within the context of the present disclosure, gene expression data may also be referred to as transcriptomics data.

The gene expression levels determined may be considered to provide a gene expression profile. By “gene expression profile” is meant a set of data relating to the level of expression of one or more of the relevant genes in a cell, in a form which allows comparison with comparable expression profiles (e.g. from cells for whom the cell type is already known), in order to assist in the identification of the cell type of the cell.

Embodiments of the present invention relate in particular to single cell gene expression data. As such, the determination of gene expression levels may involve determining the presence or amount of mRNA in a sample of one or more cells, such that the presence or amount of mRNA in each cell can be determined individually. Methods for doing this are well known to the skilled person. Single cell gene expression levels may be determined in a sample of cells using any conventional method, for example using single cell RNA sequencing (scRNAseq or scRNA-seq) or single cell quantitative PCR (sc-qPCR).

Single cell RNA sequencing typically involves a series of steps including single cell isolation (e.g. using micromanipulation, fluorescence activated cell sorting (FACS), laser capture microdissection, microfluidic technology, antibody coated magnetic particle capture, etc.), single cell library preparation (in which single cells are lysed, RNA is reverse transcribed to generate cDNAs including cell-specific barcodes—typically within a single cell droplet, and cDNAs are amplified), and sequencing (which can include 5′ end sequencing, 3′ end sequencing and/or sequencing of unique molecular identifiers or barcodes introduced in the reverse transcription step). Protocols for single cell RNA sequencing protocols may differ in the way each of the cell isolation, library preparation and sequencing steps performed. A variety of single cell RNA sequencing technologies are available, all of which may be used within the context of the present invention. For example, references to scRNAseq data may refer to data that has been acquired using any of the following protocols: Drop-Seq (Macosko et al., 2015), 10x Genomics' Chromium technology, GemCode (Zheng et al., 2017) technology, Tang et al. (2009), STRT (Islam e al., 2011), SMART-seq (Ramskold et al., 2012), CEL-seq (Hashimshony et al., 2012), RAGE-seq (Singh et al., 2019), Quartz-seq (Sasagawa et al., 2013), and Cl-CAGE (Kouno et al., 2019).

Single cell quantitative PCR typically involves a series of steps including single cell isolation (e.g. using microfluidic technologies, single cell printing, flow cytometry, etc.), followed by cell lysis and amplification of target gene expression products using gene specific primers. Genes whose expression is expected to be constant in the experimental conditions (also referred to as “housekeeping genes”) are commonly used for normalisation. Fluorescent dyes are used as reporter molecules to monitor the amplification, from which the initial quantity of the target gene expression products can be inferred.

Gene expression levels may be compared with the expression levels of the same genes in cells whose cell type is known. The single cell data to which the comparison is made may be referred to as the ‘reference data’. Accordingly, the determined gene expression levels may be compared to the expression levels in a reference group of cells with known cell types. The cells used to generate the reference data may be obtained from the same tissue type as the cell under analysis. For example, if the expression is being determined for a peripheral blood mononuclear cell, the expression levels may be compared to the expression levels in cells that are also peripheral blood mononuclear cells. Alternatively, the cells used to generate the reference data may be obtained from one or more tissue types that are not the same as the cell under analysis. For example, if the expression is being determined for a peripheral blood mononuclear cell, the expression levels may be compared to the expression levels in cells that are not peripheral blood mononuclear cells (e.g. the cells used to generate the reference data may be cord blood mononuclear cells of lymphoid tissue cells). Further, the cells used to generate the reference data may be obtained from one or more tissue types, including but not limited to the tissue type from which the cell under analysis was obtained. For example, if the expression is being determined for a peripheral blood mononuclear cell, the expression levels may be compared to the expression levels in a population of cells that comprises peripheral blood mononuclear cells, cord blood mononuclear cells, and lymphoid tissue cells.

### Protein Expression

Reference to determining the expression level of a protein refers to determination of the expression level of a protein once translated and processed, if applicable. A protein can be intracellular or extracellular. As such, within the context of the present disclosure, protein expression data may also be referred to as proteomics data.

The protein expression levels determined may be considered to provide a protein expression profile. By “protein expression profile” is meant a set of data relating to the level of expression of one or more of the relevant proteins in a cell, in a form which allows comparison with comparable expression profiles (e.g. from cells for whom the cell type is already known), in order to assist in the identification of the cell type of the cell.

Embodiments of the present invention relate in particular to single cell protein expression data. As such, the determination of protein expression levels may involve determining the presence or amount of one or more proteins in a sample of one or more cells, such that the presence or amount of the one or more proteins in each cell can be determined individually. Methods for doing this are well known to the skilled person. Single cell protein expression levels may be determined in a sample of cells using any conventional method, for example using multi-modal single cell RNA sequencing (e.g. 3′ Feature barcoding from 10X Genomics, CITE-seq, etc.) or FACS.

### Matched Gene and Protein Expression

References to determining matched protein and gene expression profiles refer to determination of a gene expression profile and a protein expression profile for one or more single cells or populations of cells, such that the protein expression profile and the gene expression profile can both be associated to the specific single cell or population of cells from which they were derived. In other words, both types of profiles can be matched to the same cell or population of cells (rather than necessarily matched to each other). In particular, matched gene and protein expression profiles may in some examples (but do not necessarily) comprise information about transcripts and proteins that are related to each other. Matched single cell gene expression and protein expression data can be obtained from combined single cell transcriptomics and proteomics protocols. In particular, matched single cell gene expression and protein expression data can be obtained from any multi-modal sequencing platform (e.g. 3′ feature barcoding from 10X Genomics, CITE-Seq, etc.). Matched single cell gene expression and protein expression data can also in some examples be obtained from protocols that combine a proteomics detection step (e.g. FACS) and a transcriptomics detection set (e.g. scRNAseq). A multi-modal single cell sequencing platform as used herein refers to a platform that measures expression of one or more proteins and one or more genes through a single nucleic acid sequencing step. By contrast, a single cell platform or protocol that combines a proteomics detection step and a transcriptomics detection step measures expression of one or more proteins and one or more genes using separate detection steps (such as e.g. detection of fluorescent tags for the proteomic step followed by sequencing of the transcriptome of the cell), where the results of the separate detection steps for a single cell can be mapped to the cell.

Methods for Classification and Clustering Based on Gene and/or Protein Expression

In some embodiments, the present invention provides methods for classifying cells in cell type classes. In particular, data obtained from analysis of gene expression and/or protein expression may be evaluated using one or more pattern recognition algorithms. Such analysis methods may be used to form a predictive model, which can be used to classify test data. For example, one convenient and particularly effective method of classification employs multivariate statistical analysis modelling, first to form a model (a “predictive mathematical model”) using data (“modelling data”) from cells of known subgroup (e.g., from cells with a known subtype), and second to classify an unknown cell according to subgroup. Such analysis methods may also be used to identify populations in a training data set, which can be used to train a predictive model. For example, one convenient and particularly effective method of classification employs unsupervised learning (e.g. clustering), to identify subpopulations in a training data set, and second to assign a class label for training purposes (a “ground truth” class assignment) to every cell in the population using data (“ground truth data”) from cells of known subgroup or from markers known to be associated with a subgroup.

Pattern recognition methods have been used widely to characterize many different types of problems ranging, for example, over linguistics, fingerprinting, chemistry and psychology. In the context of the methods described herein, pattern recognition is the use of multivariate statistics, both parametric and non-parametric, to analyse data, and hence to classify samples (or individual cells, in the present case) and to predict the value of some dependent variable (e.g. which can be a nominal variable such a cell type) based on a range of observed measurements (also referred to as predictor variables). There are two main approaches. One set of methods is termed “unsupervised” and these simply reduce data complexity in a rational way and also produce display plots which can be interpreted by the human eye. The other approach is termed “supervised” whereby a training set of samples with known class or outcome is used to produce a mathematical model which is then evaluated with independent validation data sets. Here, a “training set” of gene expression data is used to construct a statistical model that predicts correctly the “subgroup” of each sample. This training set is then tested with independent data (referred to as a test or validation set) to determine the robustness of the computer-based model. These models are sometimes termed “expert systems,” but may be based on a range of different mathematical procedures such as support vector machine, decision trees, k-nearest neighbour and naive Bayes classifiers. Supervised methods can use a data set with reduced dimensionality (for example, the first few principal components), but typically use unreduced data, with all dimensionality. In all cases the methods allow the quantitative description of the multivariate boundaries that characterize and separate each subtype in terms of its intrinsic gene or protein expression profile. It is also possible to obtain confidence limits on any predictions, for example, a level of probability to be placed on the goodness of fit (confidence in the assignment of a sample to a class). The robustness of the predictive models can also be checked using cross-validation, by leaving out selected samples from the analysis.

“Support Vector Machine” (“SVM”) is a machine learning algorithm to classify data. Given a set of training examples, each marked for belonging to one of two categories, an SVM training algorithm builds a model that assigns new examples into one category or the other. An SVM model is a representation of the examples as points in space, mapped so that the examples of the separate categories are divided by a clear gap that is as wide as possible. New examples are then mapped into that same space and predicted to belong to a category based on which side of the gap they fall on. An SVM classifier, described further herein, has been trained on gene expression profiles of cells with known cell types, and may therefore be employed to classify the gene expression profile of an unknown cell as belonging to a particular cell type or not belonging to said cell type. The output from multiple SVM classifiers may be combined to obtain a multiclass classification. In particular, each SVM may be trained to classify the gene expression profile of a cell as belonging (or not belonging) to one of a set of multiple cell types. The combined predictions from multiple such SVM classifiers may be used to provide a prediction of which of multiple cell types a cell is most likely to belong to.

“Logistic regression” is a statistical model that uses a logistic function to model a (binary or nominal) dependent variable as a function of one or more independent variables. In particular, in the simplest case of a binary dependent variable, the log odds of the binary variable being 1 vs. 0 is expressed as a linear combination of a set of independent variables, such that the logistic function can be used to determine the probability of the binary variable being 1 as a function of the dependent variables. In the context of the present disclosure, the log odds of a cell belonging to a particular cell type vs. not belonging to a particular cell type can be expressed as a linear combination of a gene expression profile of the cell type, where the parameters of the linear combination can be estimated through linear regression. A “logistic regression classifier” is a machine learning algorithm that provides a classification based on the output of a logistic regression model. For example, a logistic regression classifier may assign a class to an observation depending on whether the probability of the observation belonging to the class is above or below a threshold. Where the dependent variable is nominal (i.e. comprising multiple categories that cannot be ordered in a meaningful way), then the logistic regression is referred to as “multinomial logistic regression” and the corresponding classifier may be a “multinomial regression classifier”. A multinomial logistic regression models the probability of an observation belonging to a particular category in view of the characteristics of the observation (values of the independent variables).

A “decision tree”, also referred to as “classification and regression tree” (CART), is a model that predicts the value of a target variable (e.g. a class—in which case the decision tree may be referred to as a “classification tree”, or a real number—in which case the decision tree may be referred to as “regression tree”) based on one or more input variables as a tree structure with internal nodes labelled with an input feature, and leaf nodes labelled with a value of the target variable, a class or a probability distribution over the classes. A “decision tree ensemble” is a machine learning algorithm to classify data that combines multiple decision trees. Decision tree ensembles include random forests (where multiple trees are used to obtain a consensus decision as the mode of the classes (for classification trees) or the mean prediction (for regression trees) of the individual trees) and boosted trees (where an ensemble is incrementally built such that each new instance emphasises the training instances previously poorly captured).

An “unsupervised learning” method is a machine learning method that looks for patterns in a data set with no pre-existing labels. Unsupervised learning may make a limited set of (or no) assumptions about the data, such as e.g. the expected distribution of the data or the number of clusters. Unsupervised learning includes dimensionality reduction techniques such as principal component analysis, and clustering techniques. Clustering refers to the process of grouping or segmenting data sets with shared attributes. In other words, clustering typically aims to identify subgroups (also referred to as “clusters”) within a data set, where the data points in a subgroup are more similar to each other than they are to data in other subgroups. Clustering does not rely on data that has been labelled, classified or categorised, although labels, categories or classes can be assigned to clusters after the clusters have been identified. Various types of clustering methods are known in the art. For example, a clustering method may be a linkage based clustering (also referred to as connectivity-based clustering e.g. hierarchical clustering) which connects data that are close to each other, a centroid based clustering (e.g. k-means) that represents clusters using a single representative vector, a distribution-based clustering (e.g. Gaussian mixture models) that represents clusters using statistical distributions, a density-based clustering which defines clusters as connected dense regions in the data space, a graph-based clustering (e.g. clique analysis) which represents data points as nodes and similarity as edges and identifies structures such as cliques (a subset of nodes in a graph such that every two nodes in the subset are connected by an edge), or an unsupervised neural network (e.g. a self-organising map).

“Translation” of the descriptor coordinate axes (i.e. the single cell gene expression profile for a set of candidate descriptor variables, from which a set of predictor genes is extracted) can be useful. Examples of such translation include normalization and mean-centring. “Normalization” may be used to remove sample-to-sample (cell to cell) or gene-to-gene variation. Some commonly used methods for calculating normalization factor include: (i) global normalization that uses all genes in a single cell transcriptomics data set; (ii) housekeeping genes normalization that uses constantly expressed housekeeping/invariant genes; and (iii) internal controls normalization that uses known amount of exogenous control genes added during sequencing or single cell digital RT-PCR. In one embodiment, the genes listed in Table 2 can be normalized using the scran method as described in Lun et al., 2016. Any normalisation method that can be used to normalise single cell transcriptomics data may be sued within the context of the present disclosure.

“Mean-centring” may also be used to simplify interpretation for data visualisation and computation. Usually, for each descriptor, the average value of that descriptor for all samples is subtracted. In this way, the mean of a descriptor coincides with the origin, and all descriptors are “centred” at zero. In “unit variance scaling,” data can be scaled to equal variance. Usually, the value of each descriptor is scaled by 1/StDev, where StDev is the standard deviation for that descriptor for all samples. “Pareto scaling” is, in some sense, intermediate between mean centring and unit variance scaling. In pareto scaling, the value of each descriptor is scaled by 1/sqrt(StDev), where StDev is the standard deviation for that descriptor for all samples. In this way, each descriptor has a variance numerically equal to its initial standard deviation. The pareto scaling may be performed, for example, on raw data or mean centred data.

“Logarithmic scaling” may be used to assist interpretation when data have a positive skew and/or when data spans a large range, e.g., several orders of magnitude. Usually, for each descriptor, the value is replaced by the logarithm of that value. In “equal range scaling” (also referred to herein as “scaling” or “scaling between 0 and 1”) each descriptor is divided by the range of that descriptor for all cells in a single cell transcriptomics data set. In this way, all descriptors have the same range, that is, 1. In “autoscaling,” each data vector is mean centred and unit variance scaled. This technique can be useful because each descriptor is then weighted equally, and large and small values are treated with equal emphasis.

When comparing data from multiple analyses (e.g., comparing expression profiles for one or more test samples to the centroids constructed from samples collected and analysed in an independent study), it is advantageous to normalize data across these data sets.

“Feature selection” as used herein refers to the process of automatically identifying variables that contribute most to the prediction from a machine learning model. Once a variable is included in a machine learning model (whether or under evaluation) it may be referred to as a “feature” or “predictive feature”. Feature selection therefore refers to any process through which a subset of variables in a data set are selected to be included in a predictive model. The underlying assumption behind feature selection is that the data contains some variables that are either redundant or irrelevant. For example, when the machine learning model is a classifier, a variable may be irrelevant if the value of the variable does not associate with the classes that the classifier is designed to discriminate between. For example, the distribution of the values for the variable may not be significantly different between the different classes.

In a simple example where two classes are used and the variables are binarised gene expression values, gene expression of a particular gene may be an irrelevant variable if the cells in one class are not more likely to express (or not express) the gene than the cells in the other class. A variable may be redundant if it provides information that is already provided by another variable. For example, when the machine learning model is a classifier, a variable may be redundant if the variable is highly correlated with a feature (i.e. another variable) used by the classifier such that knowing the value of the variable does not further improve the ability of the classifier to distinguish between classes. A variable may be selected as a feature of a model if inclusion of the variable as a feature of the model improves the performance of the model. Preferably, a variable is selected as a feature of the model if it significantly improves the performance of the model. Significance may be assessed in view of the increased complexity associated with adding a new feature to the model, in order to reduce the risk of overfitting the data. Feature selection advantageously reduces the size of a model as only a subset of the variables are included as features of the model. This may make the application of the resulting model to predict characteristics of unseen data more computationally efficient. Feature selection typically comprises comparing the performance of a model with and without a feature. Many methods of feature selection are known in the art. These may differ in terms of how they select features to be assessed for inclusion in the model, how they assess the performance of the model, how they control for overfitting (i.e. how they penalise for complexity of a model), etc. All feature selection approaches known in the art that are suitable for selecting features for a classifier may be used within the context of the present disclosure.

In some cases, feature selection may be performed as part of a method of training a classification algorithm. For example, feature selection may be performed as part of a tree boosting process (i.e. as part of the process of training a boosted tree classifier), also referred to as gradient boosting applied to CARTs. Gradient bosting is a machine learning technique for regression and classification problems, in which a predictive model is built as an ensemble of weak prediction models (such as decision trees, i.e. CARTs). The technique comprises gradually building a model by including further weak prediction models, where at each step a new weak prediction model is included which focuses on capturing instances that are poorly captured by the existing ensemble model. In the context of gradient boosted trees, the weak prediction models are typically fixed size decision trees (i.e. trees with a fixed number of terminal nodes, typically between 2 and 10). The present inventors have found trees with 8 terminal nodes (3 splits) to be particularly useful in the context of identification or predictor genes that differentiate between 4 to 8 cell types.

### Methods for Identification of Cell-Type Predictive Features

In some embodiments, the present invention provides methods of identifying a predictor set of genes for predicting the cell type of one or more cells (also referred to as cell-type predictive features). Cell type predictive features or predictor genes are genes whose expression (at the transcript level) can be used to predict the cell type of a cell. In embodiments, a predictor gene is a gene that is more likely to be expressed in one or more first cell types than in one or more other cell types. As such, a single cell that is found to express the gene is more likely to belong to the one or more first cell types than the one or more other cell types.

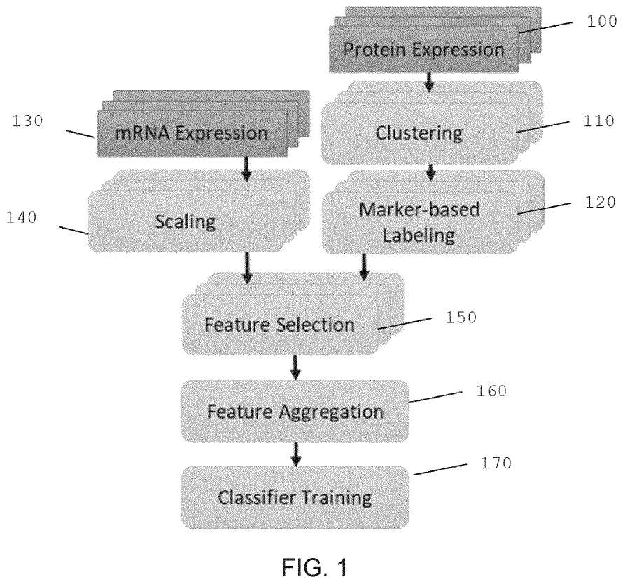

Methods of identifying a predictor set of genes as described herein use as input single cell gene expression profiles for a plurality of cells, and single cell protein expression profiles for the same plurality of cells (see FIG. 1, steps 100, 130). In other words, the methods use as input one or more data sets that comprise matched single cell transcriptomics and proteomics data. The proteomics and transcriptomics data may be independently pre-processed (see FIG. 1, step 140), for example including normalisation, scaling and/or discretisation. The proteomics data is then used to assign a cell type class to at least some of the plurality of cells. This includes using an unsupervised learning method to group cells into clusters that are assumed to have the same cell type label (see FIG. 1, step 110). The cell type label may be assigned using one or more rules that apply to the protein expression profiles of the cells in a group (see FIG. 1, step 120). For example, a group that contains at least x% of cells (where x can be e.g. 30, 40, 50, 60, 70, 80 or 90%) expressing a specific protein marker (or combination of protein markers) may be assigned a corresponding cell type label. For example, any of the following combination of cell type labels and protein markers may be used: non-CD8+ T lymphocytes—CD3, CD4; CD8+ T lymphocytes—CD3, CD+; B lymphocytes—CD19; natural killer cells—CD56; monocytes—CD14. The cell type classes assigned to the cells are then used as ground truth in a feature selection process that uses the single gene expression profiles as candidate predictive features (see FIG. 1, step 150). The feature selection process results in the identification of a predictive set of genes. These can be used to classify an unknown cell between classes that correspond to the cell type classes used in the feature identification process (see FIG. 1, steps 160-170).

### Genes Making up the Cell-Type Identification Panel or Gene Expression Profile

In accordance with any aspect of the present invention, the genes that make up the cell type identification panel/gene expression profile may be selected from any 4, 4, 5, 6, 7 or more (such as all of the) genes selected from the following group:CST3 (1471), CD8B (926), IL7R (3575), KLRF1 (51348), MS4A1 (931), CD8A (925), TRBC2 (28638), CD3D (915), GZMK (3003),

NKG7(4818), CD79A (973), TYROBP (7305), IL32 (9235), CD3E (916), CTSW (1521), GZMB (3002), CD4 (920), CD69 (969), and TRAC (28755), the number in brackets following each gene name being the NCBI Gene ID number for that gene; the nucleotide sequence for each gene as disclosed at that NCBI Gene ID number on 16 Apr. 2020 is expressly incorporated herein by reference. In some cases the CST3 gene expression is of the transcript provided under any of RefSeq ID numbers NM_000099.4 and NM_001288614.2. In some cases the CD8B gene expression is of the transcript provided under any of RefSeq ID numbers NM_001178100.1, NM_004931.5, NM_172101.4, NM_172102.4, and NM_172213.4. In some cases the IL7R gene expression is of the transcript provided under any of RefSeq ID numbers NM_002185.5, NR_120485.3 and XM_005248299.4. In some cases the KLRF1 gene expression is of the transcript provided under any of RefSeq ID numbers NM_016523.3, NM_0 NR_159359.1 01291822.2, NM_001291823.2, NM_001366534.1, NR_120305.2, NR_159360.1, NR_159361.1, XM_017019415.1 and XR_931301.2. In some cases the MS4A1 gene expression is of the transcript provided under any of RefSeq ID numbers NM_152866.3, NM_021950.3 and NM_152867.2. In some cases the CD8A gene expression is of the transcript provided under any of RefSeq ID numbers NM_001768.7, NM_171827.3, NM_001145873.1, and NR_027353.1. In some cases the CD3D gene expression is of the transcript provided under any of RefSeq ID numbers NM_000732.6 and NM_001040651.2. In some cases the GZMK gene expression is of the transcript provided under RefSeq ID number NM_002104.3. In some cases the NKG7 gene expression is of the transcript provided under any of RefSeq ID numbers NM_005601.4, NM_001363693.2, XM_005258955.3, or XM_006723228.3. In some cases the CD79A gene expression is of the transcript provided under any of RefSeq ID numbers NM_001783.4 and NM_021601.4. In some cases the TYROBP gene expression is of the transcript NM_001173514.2 provided under any of RefSeq ID numbers NM_003332.4, NM_198125.3, NM_001173514.2, NM_001173515.2 and NR_033390.2. In some cases the IL32 gene expression is of the transcript provided under any of RefSeq ID numbers NM_001376923.1, NM_004221.7, NM_001012636.2, NM_001012633.4, NM_001012631.4, NM_001012632.3, NM_001012634.4, NM_001012635.4, NM_001012718.4, NM_001308078.4, NM_001369588.3, NM_001369587.3, NM_001369589.3, NM_001369590.3, NM_001369591.3, NM_001369592.3, NM_001369593.3, NM_001369595.3, and NM_001369596.3. In some cases the CD3E gene expression is of the transcript provided under RefSeq ID number NM_000733.4. In some cases the CTSW gene expression is of the transcript provided under RefSeq ID number NM_001335.4. In some cases the GZMB gene expression is of the transcript provided under any of RefSeq ID numbers NM_004131.6, NM_001346011.2, and NR_144343.2. In some cases the CD4 gene expression is of the transcript provided under any of RefSeq ID numbers NM_000616.5, NM_001195014.3, NM_001195015.3, NM_001195016.3, NM_001195017.3, and XM_017020228.2.In some cases the CD69 gene expression is of the transcript provided under RefSeq ID number NM_001781.2. The nucleotide sequence for each transcript as disclosed at that RefSeq ID number on 16 Apr. 2020 is expressly incorporated herein by reference.

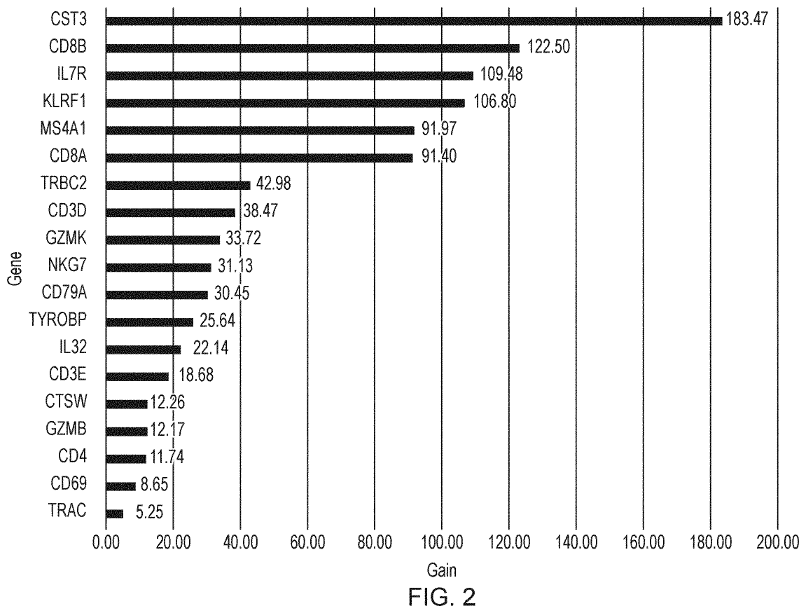

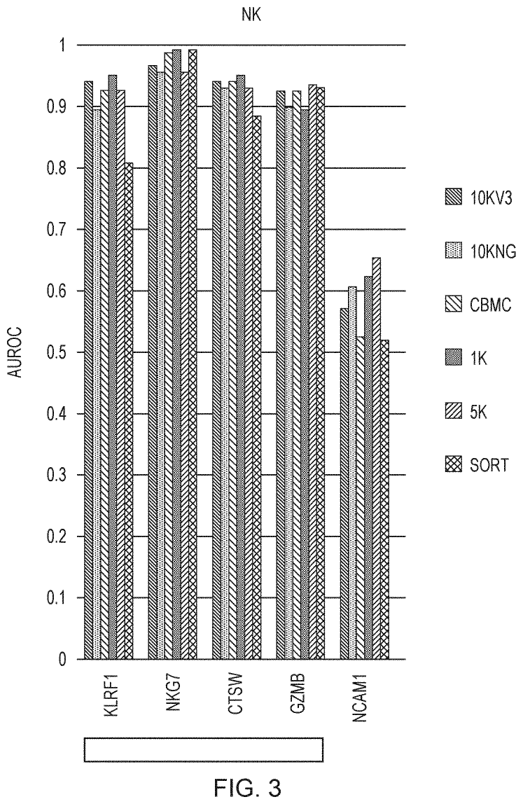

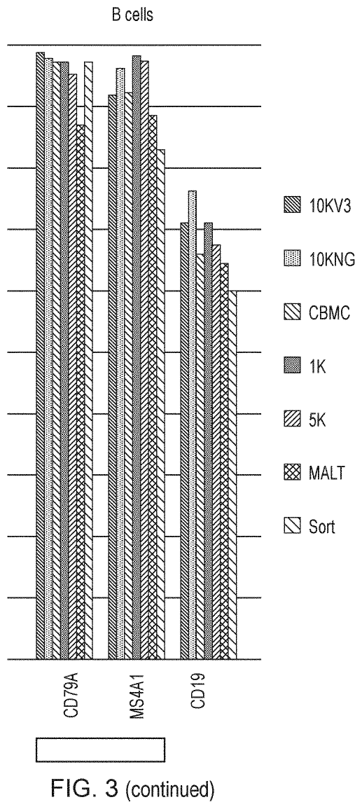

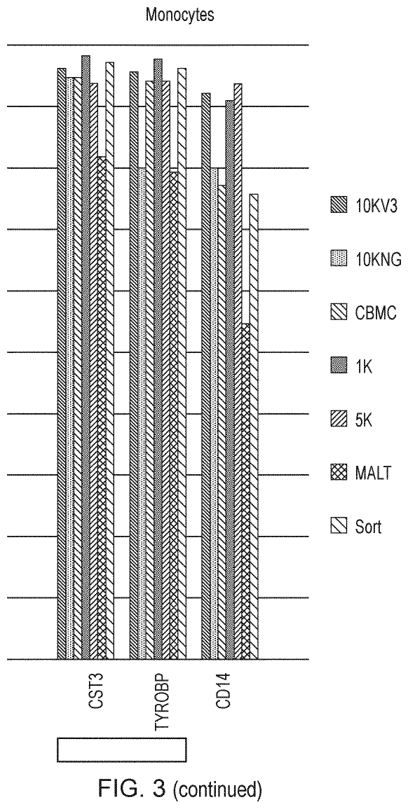

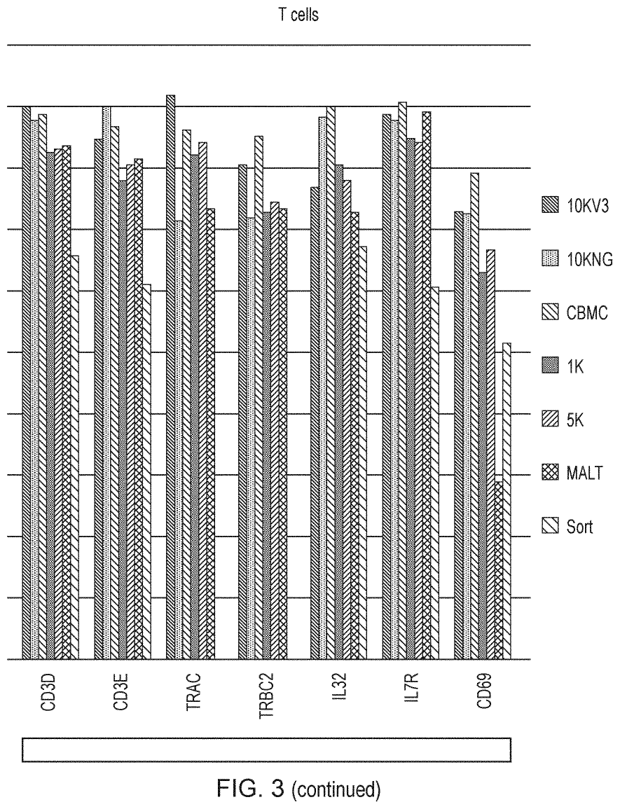

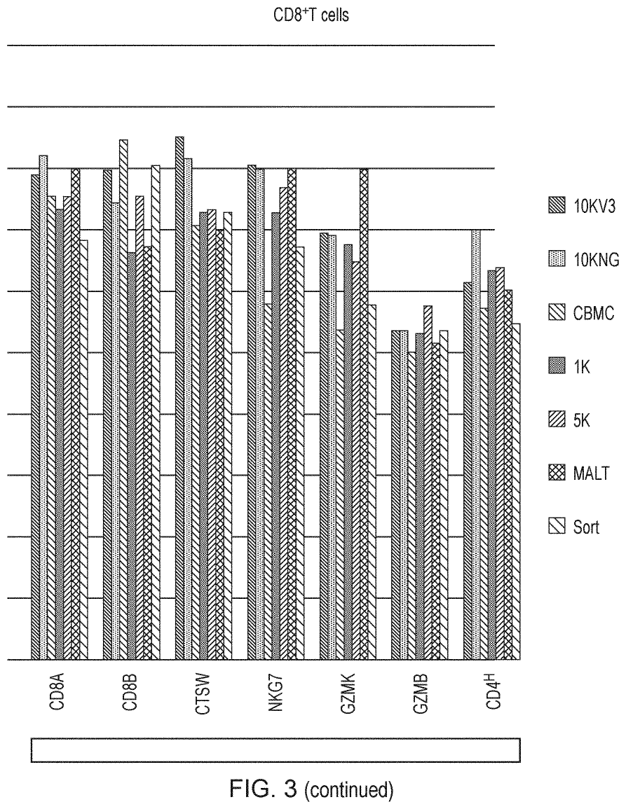

Particular subsets of the said genes are contemplated herein. For example, the genes CST3, CD8B, IL7R, KLRF1 and MS4A1, show the highest contribution to the accuracy of a classifier trained to classify scRNA expression profiles between cell types while including at least one gene that is a strong univariate predictor of classification in each of the cell types investigated, as shown in FIGS. 2 and 3. Therefore said genes may provide a compact panel of genes whose expression is significantly associated with cell type (where a cell expressing CST3 may be predicted to be a monocyte cell, a cell expressing CD8B may be predicted to be a CD8+ T cell, a cell expressing IL7R may be predicted to be a non CD8+ T cell, a cell expressing KLRF1 may be predicted to be a NK cell and a cell expressing MS4A1 may be predicted to be a B cell). In this subset, CD8B could be replaced or complemented with CD8A, and IL7R could be replaced or complemented with TRBC2 and/or CD3D with no or minimal loss of accuracy (in the case of a replacement). Similarly, a good accuracy of cell type assignment could still be achieved by replacing CST3 with TYROBP, CD8B with any of CSTW, NKG7, GZMK, GZMB, and CD4, IL7R with any of CD3E, TRAC, IL32, and CD69, KLRF1 with any of NKG7, CTSW, and GZMB, and MS4A1 with CD79A.

The following is presented by way of example and is not to be construed as a limitation to the scope of the claims.

### EXAMPLES

### Materials and Methods

### Data Sets

Three data sets comprising RNA sequencing data and cell surface marker protein expression data for single cells were used as training data. A further four data sets also including RNA sequencing data and marker protein expression data for single cells were used for validation. The characteristics of these datasets are shown in Table 1 below.

TABLE 1
Features of the data sets used in the examples. PBMC = peripheral
blood mononuclear cells, CBMC = cord blood mononuclear cells,
MALT = mucosa-associated lymphoid tissue lymphoma.

| | | | |
|---|---|---|---|
| Data set name | Number of cells | Tissue of origin | Platform |

Training data

| | | | |
|---|---|---|---|
| PBMC10KV3 | 7865 | PBMC | 10× 3′ Feature |
| | | | Barcoding |
| PBMC10KNG | 8258 | PBMC | 10× 3′ Feature |
| | | | Barcoding |
| CBMC (Stoeckius | 8617 | CBMC | CITE-Seq |
| et al., 2017) | | | |

Validation data

| | | | |
|---|---|---|---|
| PBMC1K | 713 | PBMC | 10× 3′ Feature |
| | | | Barcoding |
| PBMC5K | 5427 | PBMC | 10× 3′ Feature |
| | | | Barcoding |
| MALT | 8412 | MALT | 10× 3′ Feature |
| | | | Barcoding |
| PBMCSort (Zhang | 85423 | PBMC | FACS cell |
| et al., 2017) | | | sorting & 10× |

The PBMC10KV3, PBMC10KNG, PBMC1K, PBMC5K and MALT data sets are available from 10X Genomics at https://support.10xgenomics.com/single-cell-gene-expression/datasets (respectively https://support.10xgenomics.com/single-cell-gene-expression/datasets/3.0.0/pbmc 10k protein v3,/3.0.0/vdj v1 hs pbmc2 5gex protein, /3.0.0/pbmc 1k protein v3, 3.1.0/5k_pbmc_protein_v3, and /3.0.0/malt_10k_protein_v3).

For the PBMCSort data set, single cell RNA expression data was obtained using 10x genomics sequencing and cell surface protein marker expression data was obtained using fluorescence activated cell sorting (FACS), as explained in Zhang et al., 2017. Briefly, populations of immune cells were purified using FACS then single cell RNA expression in each cell was analysed using the Gemcode platform from 10x Genomics (whereby single cells are encapsulated in gel beads in a microfluidic chip, the gel beads containing oligonucleotides for reverse transcription of polyadenylated RNAs, generating cDNAs including a bead-specific barcode and a unique molecular identifier (UMI)—the cDNAs are subsequently pooled for amplification and sequencing library preparation).

For the CBMC data set, combined RNA sequencing and quantification of cell surface protein marker expression was obtained using CITE-Seq (Cellular Indexing of Transcriptomes and Epitopes by Sequencing) as explained in Stoeckius et al., 2017. Briefly, CITE-Seq uses antibody nucleotide conjugates (antibodies labelled with barcoding oligonucleotides) to obtain information about the presence of protein markers bound by the antibodies, via subsequent scRNA sequencing (using any known method such as e.g. Gemcode from 10x Genomics as described above or Drop-seq (which uses encapsulation of single cells together with barcoded beads that capture the cell's mRNA and on which reverse transcription can be performed to obtain barcoded cDNAs that can be processed as above)).

For all other datasets, combined RNA sequencing and quantification of cell surface protein marker expression was obtained using the 3′ Feature Barcoding technology from 10X Genomics (as explained in the protocols associated with these data sets from 10X Genomics, on the above-mentioned web-pages). Briefly, the platform combines antibody nucleotide conjugates as explained above (to quantify protein cell surface marker expression) with the 10X genomics single cell RNA sequencing approach from 10x Genomics (Gemcode).

It is important to note that the 3 datasets used for training were obtained from different tissues (PBMC and CBMC) and using different platforms (CITE-seq and 10x Genomics 3′ barcoding).

### “Ground Truth” Cell Type Classification

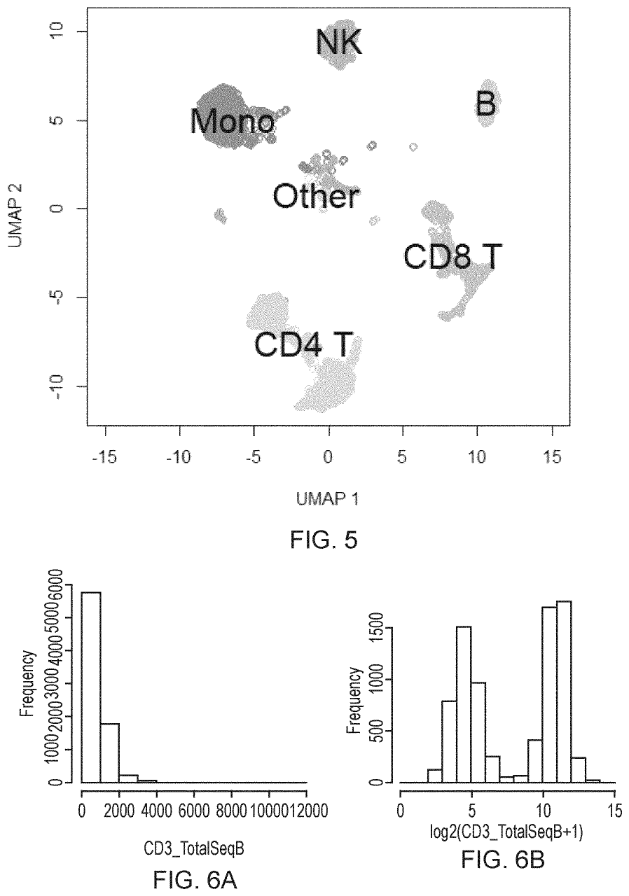

The “ground truth” cell type classification for each cell in each of the data sets was obtained based on the protein cell surface marker expression data (see FIG. 1, steps 100-120). In particular, information about the following markers was available for each dataset: CD3, CD8, CD4, CD14, CD19, CD56. This information was visualised using the UMAP algorithm (Becht et al., 2019). Brifely, the UMAP (Uniform Manifold Approximation and Projection for Dimension Reduction) algorithm performs a dimension reduction process that assumes that the data is distributed on a Riemannian manifold that can be modelled with a fuzzy topological structure, and searches for a low dimensional projection of the data that has the closest possible equivalent fuzzy topological structure. The UMAP algorithm has been widely used to visualise FACS data, where it can help to identify clusters of cells based on expression of protein markers. UMAP was used to inspect how the cells grouped together by protein expression. This analysis revealed that the PBMC cells clustered into 6 groups that can be associated with known cell types (as shown on FIG. 5 which shows the output of the UMAP algorithm for the PBMC10KV3 data set), with protein markers expression (in particular markers CD3, CD8, CD4, CD14, CD19, CD56) highly specific to the corresponding cell type. In particular, the following classes were identified:
non-CD8+ T lymphocytes: CD3+CD4+CD8− or CD3+CD25+ or CD3+CD4+CD8+ (i.e. any cluster that fell into one of these three categories was annotated as non-CD8+ T lymphocytes);
CD8+T lymphocytes: CD3+CD8+CD4− or CD3+CD8-CD4−;
B lymphocytes: CD19+;
natural killer cells: CD56+CD3−;
monocytes: CD14+CD3− or CD4+CD14-CD3− or CD15+CD16+CD14-CD3-;
other cells (including cells whose cell type cannot be determined using the above-mentioned 6 marker proteins, cells classified as progenitor cells (CD34+) in the CBMC data set, and doublets—i.e. data that represents a mixture of transcriptomes and protein expression markers from two cells trapped in the same droplet during the single cell multi-modality sequencing process).

Note that the UMAP visualisation revealed that CD4+CD8+ cells were closer to CD4+ cells than to CD8+, hence their inclusion in the “non CD8+ T lymphocytes” class. Conversely, CD8-CD4− T cells were found to be closer to the CD8+ T cells in the UMPA visualisation, and were therefore classified as CD8+ T lymphocytes.

In particular, for all data sets other than PBMCSort, the following process was used:
i. the protein expression data was log transformed (for each protein expression data point x (in UMI counts), the following quantity was obtained: log2(x+1)); the inventors found that the log transformed single cell protein expression data showed a bimodal distribution with a clear separation of cells that can be considered to be positive and negative for expression of a marker (compare FIGS. 6A and 6B showing histograms for the raw and log-transformed data for CD3 in the PBMC10KV3 data set), thereby easing the cell type assignment process;
ii. in each data set, populations of cells were identified by applying the PhenoGraph clustering algorithm (Levine et al., 2015) to the log transformed protein expression data, as implemented in the R package Rphenograph; briefly, the PhenoGraph algorithm was developed for unsupervised identification of cell populations from single cell protein marker data (mass cytometry data, in the original publication): each cell is associated with a point in high-dimensional space, with coordinates representing the expression values measured for each protein marker; a graph is built from this data by representing each cell as a node connected by a set of edges to a neighbourhood of its most similar cells, where edges are defined in a two-step process, the second step refining the connections based on the number of shared neighbours obtained in the first step, using the Jaccard similarity coefficient; sets of highly interconnected nodes (also referred to as “clusters”) are then identified from this graph as separate cell populations;
iii. the log transformed protein expression data was binarised such that for each cell and each protein marker a Boolean value (marked expressed/not-expressed) is obtained; in particular, for each data set and protein marker, a threshold was empirically chosen by inspection of the distribution of log transformed protein expression values and UMAP visualisation; as all log transformed protein expression value distributions were bimodal, the valley between the two modes could be identified for each protein marker and used as a binarisation threshold; such a threshold can be identified, e.g. manually or automatically based on the parameters of a mixture model (e.g. a Gaussian mixture model) fitted to the data; all thresholds were found to be within a small range of each other (specifically, around 6-7) in the present data sets;
iv. each cluster identified in step ii was assigned a cell type by labelling the cluster as positive for a particular protein marker if more than 40% of the cells in the cluster express the marker; the 40% threshold was empirically chosen based on the expression levels of marker proteins in clusters identified by UMPA clustering; the process of determining an appropriate threshold was performed independently for each data set; and
v. the cluster labels were propagated to all individual cells in the respective clusters.

For PBMCSort, the ground truth class assignment was derived from the FACS data available as part of the published data set (Zheng et al., 2017).

scRNAseq Data Pre-Processing

Each scRNAseq data set was independently pre-processed. In particular, each scRNAseq data set was normalised using the method described in Lun et al., 2016, as implemented in the R package “scrap”. Briefly, this method adjusts the expression data to account for differences in library sizes derived from single cells. This is performed by repeatedly normalising expression values relative to summed expression values across pools of cells, then deconvoluting pool-based size factors to yield cell-based factors. The expression level of each gene was then scaled to a range between 0 and 1 (see FIG. 1, steps 130-140), then binarised to either 0 or 1 using a threshold of 0.01. Linear scaling was used, mapping the range of minimum to maximum expression for each gene to the [0,1] interval. Without wishing to be bound by any particular theory, it is believed that scRNA-seq data for a particular gene frequently shows a zero-inflated distribution. In particular, the transcripts associated with the gene are not detected in a first subset of cells (thereby creating a peak at 0 in the distribution of UMI counts), and are detected to various extents in a second subset of cells (forming a distribution centred around higher UMI counts, where the centre of this distribution varies depending on the gene). As such scaling the scRNA-seq data between 0 and 1 advantageously makes it relatively straightforward to define a common threshold that separates the cells in the first subset (zero peak) from the cells in the second subset, for all genes. Binarisation of the data was found to lead to models that are more robust, i.e. models that generalise well between a training data set and a validation data set. In other words, without wishing to be bound by theory, it is believed that the binarisation helps to reduce the risk of overfitting the model to the training data. The specific threshold used for binarisation was selected empirically, by inspection of histograms of the scaled expression data. Thresholds between 0.01 and 0.1 were tested, and all were found to perform satisfactorily.

As a result of the above processes, each processed data set comprised, for each cell: a binarised mRNA expression profile, and a cell type label (chosen from: “non-CD8+ T lymphocyte”, C“D8+ T lymphocyte”, “B lymphocyte”, “natural killer cell”, “monocyte”, and “other”) derived from the protein marker data—also referred to as “ground truth”. This data was used for feature selection, classification model training and validation as explained below.

### EXAMPLE 1

### End-to-End Multi-Omics Feature Selection Identified a Concise Panel for Cell-Type Assignment

In this example, the inventors showed how the training data (PBMC10KNG, PBMC10KV3, CBMC) described can be used to identify a robust set of mRNA markers for cell type assignment according to the disclosure (also referred to as “feature selection, step 150, FIG. 1).

### Methodology

The mRNA features for cell-type assignment were selected using the gradient boosted tree model implemented in the XGBoost package. The XGBoost package is an optimized distributed gradient boosting library, providing algorithms to build machine learning models in a gradient boosted framework, and in particular gradient boosted trees. For each of the data sets in the training set, a total of 100 XGBoost models were trained by subsampling 80% of the processed mRNA expression data (excluding mitochondrial and ribosomal genes), using the ground truth labels derived from the protein marker data as explained above. All the regularisation terms (alpha, gamma, and lambda) were set as 0. Regularisation terms are normally used to control overfitting by penalizing model complexity (i.e. focusing on the most stringent features). In the present case, as will be explained further below, a stringent criterion for selection of features was applied on the aggregated results from the multiple data sets. As such, the inventors chose to relax the regularization terms to capture subtle features in each individual data set. The learning rate was set to 0.05, the subsampling ratio was set to 0.5, the maximum tree depth was set to 3, and the number of trees in an ensemble was set as 100 (i.e. an ensemble model of 100 trees or maximum depth 3 was trained in each XGBoost run). These parameters were chosen using a cross validated grid search for the parameters providing highest accuracy, using the PBMC10KV3 data set. Without wishing to be bound by particular theory, the subsampling ratio, learning rate and maximum tree depth are thought to control overfitting (with high values of the subsampling ratio and maximum tree depth, and lower values of the learning rate resulting in higher risks of overfitting). In the present case, as the trees are trained to classify data between 6 categories, the inventors reasoned that a maximum tree depth of 3 (leading to 8 leaves) should have sufficient complexity. Conversely, relatively slow learning rates (as it the case here) may help to capture subtle but potentially relevant patterns. The inventors further reasoned that the stringent criterion applied to select features using the aggregated results from each data set (see below) would likely deal with any issues that may arise from subtle patterns being falsely identified as potentially relevant in individual data sets. Each run of the XGBoost optimization process produces a model that predicts a class for a single cell represented by its binarised mRNA expression profile, using a subset of the predictive features (binarised mRNA expression value for each single gene) available as part of the particular training dataset used. The feature selection process took 51281.86, 54963.73, 64606.13 seconds (in total, i.e. for all 100 runs) for PBMC10KV3, PBMC10KNG, CBMC, respectively (all using an Intel Xeon E5-2680 v3 process).

The results of the 100 runs for each of the 3 training data sets (i.e. 300 runs in total) were combined to select a robust set of features that are predictive of cell type across all of these data sets. This was performed by selecting gene expression markers that were included as predictive features in every single one of the 100 models, for at least two of the 3 training data sets. The resulting set is referred to herein as the “AMASC” (Automated Marker Analysis for Single-Cell RNA-seq) set or gene panel. Without wishing to be bound by particular theory, it is believed that this approach, which follows a principle of parsimony by selecting simple set of features that are common across experiments, would select markers that are likely to reflect true biological features of the different types of cells, and would therefore represent a robust predictive panel of genes.

Indeed, the inventors observed that some genes were expressed in a cell type in one data set but not in the other data sets. In theory, such differences between data sets could be caused by various factors such as biological conditions, tissue types, batch effects, or protocols. These differences make it very difficult to identify which genes are genuine cell type markers by looking at a single data set. Even with extensive expert curation, it is likely to be very difficult to interpret the correlation between the behaviours of specific genes and the parameters of a data set in order to decide if a gene is really a cell type marker. Further, the inventors observed that merging multiple data sets and performing the feature selection directly on such a merged data set did not directly solve this problem. Indeed, effects from a larger data set may then overwhelm smaller ones, and merging multiple data sets is likely to distort (potentially informative) behaviours of the genes in the data sets Therefore, the inventors took a rather conservative (parsimonious) approach to identify the genes that are predictive to the cell types across multiple data sets. If a gene was consistently selected as a predictive marker in all the subsets of cells from multiple training data sets, it is likely to be a marker that would be indicative of the cell type regardless of the conditions (or any other parameters of the data set that may influence the results).

The predictive value of each of the selected features was investigated in two ways. Firstly, for each selected feature (gene), the average gain in classification accuracy obtained by adding a split based on the feature to an ensemble of classifier trees that is being trained was quantified (as implemented in the function “model.get score(importance type=‘gain’” in XGBoost). In particular, an XGBoost model was trained as explained above, except using a concatenated training set including all 3 training data sets. The average Gain associated with adding splits based on each gene, to an ensemble of classifier trees that are being trained, was calculated. The Gain quantifies the difference between the classification scores for the new leaves associated with a split and the classification score for the original leaf before the split. The classification score for a leaf quantifies the accuracy of the classification, using a logistic loss function comparing the predictions from the classifier and the ground truth labels. As such, this analysis demonstrates the predictive value of each of the selected genes as part of models trained to assign cells to one of the 6 classes, using multiple genes selected from the set of selected features.

Secondly, for each of the selected features, the univariate predictive value of the feature was evaluated by quantifying the area under the receiver operating curve (AUROC) as implemented in the pROC package in R (Robin et al., 2011), separately for each cell type class. Briefly, a ROC curve is a way of analyzing the performance of a binary classification method by quantifying the sensitivity (proportion of correctly classified positive observations, i.e. true positives, TP) and specificity (proportion of correctly classified negative observations, i.e. true negatives, TN) as an output threshold is moved. The AUROC is the area under the curve of sensitivity as a function of specificity. The higher the AUROC the more accurate the binary classifier is believed to be. In the present instance, the ROC curves were calculated by quantifying TP and FP rates as the threshold for predicting a particular cell type for a cell is moved from (1) expression of the feature is “0” to (2) expression of the feature is “1” (maximal expression value observed for the gene). These values were compared with the univariate predictive values for genes that are “traditional” cell type markers at the protein level. For NK cells, B lymphocytes, T lymphocytes and monocytes, the performance of univariate models trained to predict one cell type vs. all others was evaluated. For CD8+ T, the performance was evaluated within T cells (CD8+ T and non-CD8+ T). This analysis demonstrates the predictive value of each of the selected genes individually in relation to individual cell types. In other words, genes with high univariate predictive value for a particular cell type can be used individually as reliable markers of whether a cell is likely to belong to the particular cell type or not (i.e. one vs. others classification).

As a result of the above analysis, each cell type could be associated with a set of mRNA markers whose expression is predictive of the cell type in the sense that if the gene is expressed at the transcript level then the cell is likely to belong to the said cell type, and conversely if the gene is not expressed then the cell is unlikely to belong to the cell type (or in other words, the cell is likely to belong to another cell type).

Finally, having established that each of the selected features has predictive value individually for at least one cell type, and that each of the selected features has predictive value in discriminating between all 6 cell types as part of the set of selected features, the inventors investigated whether restricted subsets of the set of selected features could still accurately discriminate between all 6 cell types. In particular, the inventors evaluated the multiclass classification accuracy for randomly selected subsets of 1-19 genes in the set of selected features. For each gene set size, 10 experiments were run (i.e. 10 different subsets of the complete set of selected features were randomly selected).

### Results

As described above, the inventors applied the feature selection workflow on three data sets of different platforms and tissues of origin to identify the features that are predictive of the five common immune cell types. Although each run of the feature selection identified a large number of genes (median gene numbers—calculated as the average of the 50^{th} and the 51^{st} numbers of the 100 runs: 271, 277.5, 416 genes for PBMC10KNG, PBMC10KV3, CBMC, respectively), the intersection of the genes that were frequently selected in all the feature selection runs was small. Using a strict criterion for feature selection (as explained above), a panel of 19 genes was identified (referred to as the “AMASC” genes). The AMASC genes and their importance in the trained XGBoost model are shown in FIG. 2. They include (GeneID numbers in brackets): CST3 (1471), CD8B (926), IL7R (3575), KLRF1 (51348), MS4A1 (931), CD8A (925), TRBC2 (28638), CD3D (915), GZMK (3003), NKG7(4818), CD79A (973), TYROBP (7305), IL32 (9235), CD3E (916), CTSW (1521), GZMB (3002), CD4 (920), CD69 (969), and TRAC (28755).

The predictive value for each AMASC gene was assessed with AU ROC analysis as explained above. The results of this are shown in FIG. 3 and Table 2 below. In FIG. 3 and Table 2 below, “T cells” refers to a combined class comprising both CD8+and CD8− T cells, and “CD8+ T cells” refers to the univariate prediction accuracy for CD8+ vs non-CD8+ T cells, within the T cell class. The AUROC scores of CD79A and MS4A1 for B lymphocytes were above 0.95 in all but the MALT data set (AUROC 0.83). These figures were much higher than for CD19, which is a marker for B lymphocytes, and the marker used at the protein level to establish the ground truth used in training the models. For natural killer cells, the scores of GZMB, KLRF1, NKG7, CTSW were above 0.8 for all the data sets (NB: the MALT data set did not include information sufficient to identify NK cells, hence the “NAs” in Table 2). By contrast, the score of the NCAM1 gene (its protein product CD56 was the marker used to define the cell population) was less than 0.66 for the analyzed data sets. The scores of CST3 were above 0.81 in predicting monocytes, higher than those of CD14, which was the marker used at the protein level to define the cell type. For non-CD8⁺ and CD8³⁰ T lymphocytes (where these two classes are mutually exclusives such that a gene whose expression is predictive for CD8+ T lymphocytes provides complementary information for non-CD8+ T lymphocytes), CD8A, CD8B, CTSW, and NKG7 were again more predictive than CD4 for each analyzed data set. The data also suggests that CD4 is not prevalently expressed by the T cells. It is however predictive for monocytes in at least some data sets.

TABLE 2
AUROC from binary univariate predictors using binarised
expression values for each of the AMASC genes and comparative
examples (marked by *), for each of the data sets above.
NA = data for the gene or cell type was not available
in this data set. T cells = CD8+ and non-CD8+ T cells.

| | | | | | | | |
|---|---|---|---|---|---|---|---|
| Gene | 10KV3 | 10KNG | CBMC | 1K | 5K | MALT | SORT |

NK cells

| | | | | | | | |
|---|---|---|---|---|---|---|---|
| KLRF1 | 0.94 | 0.89 | 0.93 | 0.95 | 0.92 | NA | 0.80 |
| NKG7 | 0.96 | 0.95 | 0.98 | 0.99 | 0.96 | NA | 0.99 |
| CSTW | 0.94 | 0.93 | 0.94 | 0.95 | 0.93 | NA | 0.88 |
| GZMB | 0.92 | 0.90 | 0.92 | 0.89 | 0.93 | NA | 0.93 |
| NCAM1* | 0.57 | 0.60 | 0.53 | 0.62 | 0.66 | NA | 0.52 |
| TYROBP | 0.68 | 0.68 | 0.59 | 0.65 | 0.68 | NA | 0.95 |

B cells

| | | | | | | | |
|---|---|---|---|---|---|---|---|
| CD79A | 0.98 | 0.97 | 0.97 | 0.97 | 0.95 | 0.87 | 0.97 |
| MS4A1 | 0.91 | 0.96 | 0.92 | 0.98 | 0.97 | 0.88 | 0.83 |
| CD19* | 0.71 | 0.76 | 0.66 | 0.71 | 0.68 | 0.64 | 0.60 |

Monocytes

| | | | | | | | |
|---|---|---|---|---|---|---|---|
| CST3 | 0.96 | 0.94 | 0.94 | 0.98 | 0.93 | 0.81 | 0.97 |
| TYROBP | 0.95 | 0.92 | 0.94 | 0.97 | 0.94 | 0.79 | 0.96 |
| CD4 | 0.70 | 0.61 | 0.55 | 0.80 | 0.76 | 0.67 | 0.57 |
| CD14 | 0.92 | 0.80 | 0.77 | 0.91 | 0.93 | 0.54 | 0.75 |

T cells

| | | | | | | | |
|---|---|---|---|---|---|---|---|
| CD3D | 0.90 | 0.88 | 0.89 | 0.83 | 0.83 | 0.83 | 0.66 |
| CD3E | 0.85 | 0.91 | 0.87 | 0.78 | 0.81 | 0.82 | 0.61 |
| TRAC | 0.92 | 0.72 | 0.86 | 0.82 | 0.84 | 0.74 | NA |
| TRBC2 | 0.80 | 0.72 | 0.85 | 0.73 | 0.75 | 0.73 | NA |
| IL32 | 0.77 | 0.88 | 0.90 | 0.81 | 0.78 | 0.73 | 0.67 |
| IL7R | 0.89 | 0.88 | 0.91 | 0.85 | 0.84 | 0.89 | 0.61 |
| CD69 | 0.73 | 0.73 | 0.79 | 0.63 | 0.67 | 0.29 | 0.52 |

CD8+ T cells

| | | | | | | | |
|---|---|---|---|---|---|---|---|
| CD8A | 0.79 | 0.82 | 0.76 | 0.74 | 0.76 | 0.80 | 0.69 |
| CD8B | 0.80 | 0.74 | 0.85 | 0.66 | 0.76 | 0.67 | 0.81 |
| CSTW | 0.85 | 0.81 | 0.71 | 0.73 | 0.74 | 0.70 | 0.73 |
| NKG7 | 0.75 | 0.80 | 0.58 | 0.73 | 0.77 | 0.80 | 0.67 |
| GZMK | 0.69 | 0.69 | 0.53 | 0.67 | 0.65 | 0.80 | 0.58 |
| GZMB | 0.54 | 0.54 | 0.50 | 0.53 | 0.58 | 0.51 | 0.54 |
| CD4 | 0.61 | 0.70 | 0.57 | 0.63 | 0.64 | 0.60 | 0.54 |

The proportion of cells expressing each of the selected genes, in each ground truth cell type class in each data set is shown in Table 3 below.

TABLE 3
Proportion of cells in each cell type expressing each of the AMASC genes.
Genes in Table 2 are highlighted (with *) in the table below.

| | | | | | | | |
|---|---|---|---|---|---|---|---|
| Gene | 10KV3 | 10KNG | CBMC | 1K | 5K | MALT | SORT |

NK cells

| | | | | | | | |
|---|---|---|---|---|---|---|---|
| CD3D | 0.02 | 0.03 | 0.24 | 0.00 | 0.31 | NA | 0.02 |
| CD3E | 0.76 | 0.62 | 0.12 | 0.69 | 0.41 | NA | 0.06 |
| TRAC | 0.16 | 0.12 | 0.08 | 0.13 | 0.24 | NA | NA |
| TRBC2 | 0.60 | 0.68 | 0.70 | 0.53 | 0.63 | NA | NA |
| CD69 | 0.47 | 0.73 | 0.56 | 0.88 | 0.52 | NA | 0.32 |
| IL32 | 0.92 | 0.44 | 0.27 | 0.28 | 0.43 | NA | 0.23 |
| IL7R | 0.03 | 0.07 | 0.04 | 0.13 | 0.08 | NA | 0.02 |
| CD8A | 0.02 | 0.26 | 0.11 | 0.09 | 0.07 | NA | 0.06 |
| CD8B | 0.00 | 0.02 | 0.01 | 0.03 | 0.01 | NA | 0.01 |
| GZMK | 0.07 | 0.18 | 0.10 | 0.28 | 0.10 | NA | 0.10 |
| CD79A | 0.01 | 0.02 | 0.02 | 0.03 | 0.01 | NA | 0.01 |
| MS4A1 | 0.00 | 0.01 | 0.01 | 0.03 | 0.01 | NA | 0.00 |
| CST3 | 0.13 | 0.14 | 0.20 | 0.13 | 0.10 | NA | 0.03 |
| CD4 | 0.01 | 0.01 | 0.00 | 0.00 | 0.01 | NA | 0.00 |
| TYROBP* | 0.97 | 0.93 | 0.95 | 0.94 | 0.93 | NA | 0.96 |
| CTSW* | 0.98 | 0.94 | 0.92 | 0.94 | 0.93 | NA | 0.88 |
| KLRF1* | 0.91 | 0.80 | 0.88 | 0.91 | 0.87 | NA | 0.61 |
| NKG7* | 0.99 | 0.95 | 0.99 | 1.00 | 0.96 | NA | 0.99 |
| GZMB* | 0.89 | 0.82 | 0.89 | 0.81 | 0.89 | NA | 0.86 |

B cells

| | | | | | | | |
|---|---|---|---|---|---|---|---|
| CD3D | 0.01 | 0.01 | 0.05 | 0.03 | 0.08 | 0.01 | 0.00 |
| CD3E | 0.03 | 0.02 | 0.02 | 0.01 | 0.05 | 0.01 | 0.00 |
| TRAC | 0.10 | 0.12 | 0.04 | 0.12 | 0.16 | 0.09 | NA |
| TRBC2 | 0.50 | 0.64 | 0.23 | 0.48 | 0.43 | 0.21 | NA |
| CD69 | 0.57 | 0.90 | 0.83 | 0.91 | 0.82 | 0.94 | 0.28 |
| IL32 | 0.02 | 0.03 | 0.04 | 0.00 | 0.06 | 0.01 | 0.00 |
| IL7R | 0.02 | 0.02 | 0.06 | 0.01 | 0.08 | 0.02 | 0.00 |
| CD8A | 0.00 | 0.00 | 0.00 | 0.00 | 0.01 | 0.00 | 0.00 |
| CD8B | 0.00 | 0.00 | 0.00 | 0.00 | 0.00 | 0.00 | 0.00 |
| GZMK | 0.00 | 0.00 | 0.00 | 0.00 | 0.01 | 0.01 | 0.00 |
| CD79A* | 0.98 | 0.97 | 0.95 | 0.95 | 0.91 | 0.87 | 0.94 |
| MS4A1* | 0.84 | 0.94 | 0.84 | 0.97 | 0.94 | 0.92 | 0.65 |
| CST3 | 0.03 | 0.05 | 0.19 | 0.03 | 0.06 | 0.01 | 0.00 |
| CD4 | 0.01 | 0.00 | 0.02 | 0.01 | 0.02 | 0.00 | 0.00 |
| TYROBP | 0.03 | 0.05 | 0.23 | 0.01 | 0.05 | 0.01 | 0.00 |
| CTSW | 0.02 | 0.03 | 0.03 | 0.00 | 0.02 | 0.01 | 0.00 |
| KLRF1 | 0.01 | 0.01 | 0.02 | 0.00 | 0.01 | 0.00 | 0.00 |
| NKG7 | 0.03 | 0.02 | 0.20 | 0.00 | 0.05 | 0.01 | 0.00 |
| GZMB | 0.01 | 0.01 | 0.06 | 0.01 | 0.01 | 0.01 | 0.00 |

Monocytes

| | | | | | | | |
|---|---|---|---|---|---|---|---|
| CD3D | 0.01 | 0.01 | 0.05 | 0.01 | 0.05 | NA | 0.00 |
| CD3E | 0.04 | 0.02 | 0.02 | 0.01 | 0.04 | NA | 0.00 |
| TRAC | 0.02 | 0.01 | 0.02 | 0.02 | 0.04 | NA | NA |
| TRBC2 | 0.04 | 0.01 | 0.04 | 0.02 | 0.05 | NA | NA |
| CD69 | 0.08 | 0.08 | 0.09 | 0.16 | 0.10 | NA | 0.01 |
| IL32 | 0.05 | 0.02 | 0.03 | 0.02 | 0.04 | NA | 0.01 |
| IL7R | 0.02 | 0.03 | 0.02 | 0.02 | 0.05 | NA | 0.00 |
| CD8A | 0.00 | 0.00 | 0.00 | 0.01 | 0.01 | NA | 0.00 |
| CD8B | 0.00 | 0.00 | 0.00 | 0.00 | 0.01 | NA | 0.00 |
| GZMK | 0.01 | 0.00 | 0.00 | 0.00 | 0.01 | NA | 0.00 |
| CD79A | 0.04 | 0.02 | 0.02 | 0.04 | 0.01 | NA | 0.01 |
| MS4A1 | 0.04 | 0.00 | 0.01 | 0.02 | 0.02 | NA | 0.00 |
| CST3* | 0.96 | 0.97 | 0.99 | 0.97 | 0.97 | NA | 0.94 |
| CD4* | 0.53 | 0.43 | 0.19 | 0.69 | 0.70 | NA | 0.18 |
| TYROBP* | 0.98 | 0.97 | 1.00 | 0.98 | 0.96 | NA | 0.96 |
| CTSW | 0.05 | 0.03 | 0.03 | 0.02 | 0.02 | NA | 0.01 |
| KLRF1 | 0.03 | 0.00 | 0.02 | 0.01 | 0.02 | NA | 0.00 |
| NKG7 | 0.11 | 0.10 | 0.20 | 0.11 | 0.15 | NA | 0.03 |
| GZMB | 0.03 | 0.03 | 0.06 | 0.03 | 0.01 | NA | 0.00 |

Non CD8+ T cells

| | | | | | | | |
|---|---|---|---|---|---|---|---|
| CD3D* | 0.85 | 0.78 | 0.86 | 0.66 | 0.73 | 0.74 | 0.74 |
| CD3E* | 0.93 | 0.87 | 0.78 | 0.60 | 0.68 | 0.71 | 0.76 |
| TRAC* | 0.91 | 0.49 | 0.76 | 0.69 | 0.73 | 0.64 | NA |
| TRBC2* | 0.81 | 0.68 | 0.86 | 0.59 | 0.63 | 0.67 | NA |
| CD69* | 0.69 | 0.84 | 0.84 | 0.64 | 0.62 | 0.86 | 0.19 |
| IL32* | 0.87 | 0.83 | 0.88 | 0.62 | 0.64 | 0.52 | 0.63 |
| IL7R* | 0.86 | 0.82 | 0.85 | 0.72 | 0.75 | 0.86 | 0.33 |
| CD8A* | 0.02 | 0.02 | 0.01 | 0.00 | 0.01 | 0.02 | 0.00 |
| CD8B* | 0.02 | 0.02 | 0.04 | 0.00 | 0.02 | 0.02 | 0.01 |
| GZMK* | 0.13 | 0.08 | 0.01 | 0.06 | 0.12 | 0.23 | 0.04 |
| CD79A | 0.02 | 0.04 | 0.04 | 0.03 | 0.02 | 0.16 | 0.01 |
| MS4A1 | 0.01 | 0.03 | 0.01 | 0.02 | 0.01 | 0.19 | 0.00 |
| CST3 | 0.04 | 0.04 | 0.22 | 0.03 | 0.07 | 0.03 | 0.00 |
| CD4 | 0.23 | 0.41 | 0.14 | 0.27 | 0.28 | 0.22 | 0.08 |
| TYROBP | 0.05 | 0.04 | 0.22 | 0.02 | 0.07 | 0.01 | 0.00 |
| CTSW | 0.20 | 0.28 | 0.07 | 0.17 | 0.16 | 0.07 | 0.09 |
| KLRF1 | 0.02 | 0.01 | 0.03 | 0.00 | 0.02 | 0.00 | 0.00 |
| NKG7* | 0.12 | 0.06 | 0.15 | 0.04 | 0.12 | 0.06 | 0.01 |
| GZMB* | 0.02 | 0.01 | 0.06 | 0.00 | 0.02 | 0.00 | 0.00 |

CD8+ T cells

| | | | | | | | |
|---|---|---|---|---|---|---|---|
| CD3D* | 0.88 | 0.79 | 0.95 | 0.68 | 0.80 | 0.72 | 0.81 |
| CD3E* | 0.94 | 0.87 | 0.84 | 0.66 | 0.76 | 0.66 | 0.76 |
| TRAC* | 0.88 | 0.48 | 0.84 | 0.63 | 0.73 | 0.49 | NA |
| TRBC2* | 0.79 | 0.63 | 0.86 | 0.52 | 0.65 | 0.60 | NA |
| CD69* | 0.71 | 0.79 | 0.78 | 0.66 | 0.63 | 0.88 | 0.20 |
| IL32* | 0.94 | 0.88 | 0.89 | 0.67 | 0.75 | 0.52 | 0.66 |
| IL7R* | 0.77 | 0.75 | 0.81 | 0.71 | 0.58 | 0.76 | 0.31 |
| CD8A* | 0.60 | 0.67 | 0.52 | 0.48 | 0.52 | 0.61 | 0.36 |
| CD8B* | 0.61 | 0.51 | 0.72 | 0.33 | 0.53 | 0.36 | 0.68 |
| GZMK* | 0.51 | 0.45 | 0.08 | 0.40 | 0.40 | 0.80 | 0.10 |
| CD79A | 0.04 | 0.07 | 0.18 | 0.04 | 0.03 | 0.11 | 0.08 |
| MS4A1 | 0.03 | 0.04 | 0.01 | 0.02 | 0.03 | 0.15 | 0.00 |
| CST3 | 0.03 | 0.05 | 0.18 | 0.02 | 0.08 | 0.01 | 0.00 |
| CD4 | 0.00 | 0.01 | 0.00 | 0.00 | 0.01 | 0.01 | 0.00 |
| TYROBP | 0.07 | 0.19 | 0.22 | 0.11 | 0.12 | 0.01 | 0.02 |
| CTSW | 0.84 | 0.79 | 0.49 | 0.58 | 0.59 | 0.45 | 0.48 |
| KLRF1 | 0.15 | 0.08 | 0.03 | 0.04 | 0.13 | 0.03 | 0.00 |
| NKG7* | 0.60 | 0.64 | 0.31 | 0.48 | 0.63 | 0.64 | 0.18 |
| GZMB* | 0.09 | 0.08 | 0.06 | 0.06 | 0.18 | 0.03 | 0.03 |

Other

| | | | | | | | |
|---|---|---|---|---|---|---|---|
| CD3D | 0.55 | 0.26 | 0.31 | NA | 0.03 | 0.04 | NA |
| CD3E | 0.74 | 0.31 | 0.19 | NA | 0.04 | 0.10 | NA |
| TRAC | 0.58 | 0.18 | 0.16 | NA | 0.03 | 0.07 | NA |
| TRBC2 | 0.66 | 0.32 | 0.38 | NA | 0.06 | 0.04 | NA |
| CD69 | 0.54 | 0.44 | 0.52 | NA | 0.25 | 0.69 | NA |
| IL32 | 0.86 | 0.33 | 0.20 | NA | 0.05 | 0.10 | NA |
| IL7R | 0.60 | 0.30 | 0.16 | NA | 0.05 | 0.12 | NA |
| CD8A | 0.17 | 0.10 | 0.03 | NA | 0.00 | 0.01 | NA |
| CD8B | 0.10 | 0.08 | 0.02 | NA | 0.00 | 0.00 | NA |
| GZMK | 0.22 | 0.09 | 0.02 | NA | 0.00 | 0.00 | NA |
| CD79A | 0.15 | 0.13 | 0.10 | NA | 0.04 | 0.15 | NA |
| MS4A1 | 0.14 | 0.12 | 0.06 | NA | 0.01 | 0.24 | NA |
| CST3 | 0.54 | 0.87 | 0.75 | NA | 0.95 | 0.88 | NA |
| CD4 | 0.42 | 0.37 | 0.10 | NA | 0.68 | 0.57 | NA |
| TYROBP | 0.71 | 0.81 | 0.72 | NA | 0.91 | 0.78 | NA |
| CTSW | 0.53 | 0.26 | 0.33 | NA | 0.14 | 0.01 | NA |
| KLRF1 | 0.32 | 0.04 | 0.23 | NA | 0.04 | 0.00 | NA |
| NKG7 | 0.61 | 0.23 | 0.69 | NA | 0.27 | 0.09 | NA |
| GZMB | 0.28 | 0.05 | 0.39 | NA | 0.13 | 0.31 | NA |

This data on FIG. 3, and Tables 2-3 shows that the following patterns of genes expression were specific to the following cell types, and further that some of these genes are specifically and consistently expressed in these cell types (and as such can be considered to find use as single gene marker predictors for these cell types):
NK cells:
   consistently express (over 40% of cells, all data sets): TYROBP, CTSW, KLRF1, NKG7, GZMB, TRBC2, CD69;
   consistently do not express (under 40% of cells, all data sets): CD3D, TRAC, IL7R, CD8A, DC8B, GZMK, CD79A, MS4A1, CST3, CD4;
   single gene markers (high accuracy univariate predictors of NK vs. others): KLRF1, NKG7, CTSW, GZMB, TYROBP;
B cells:
   consistently express (over 40% of cells, all data sets): CD79A, MS4A1;
   consistently do not express (under 40% of cells, all data sets): CD3D, CD3E, TRAC, IL32, IL7R, CD8A, CD8B, GZMK, CST3, CD4, TYROBP, CTSW, KLRF1, NKG7, GZMB
   single gene markers (high accuracy univariate predictors of B cells vs. others): CD79A, MS4A1;
Monocytes:
   consistently express (over 40% of cells, all data sets): CST3, TYROBP, CD4;
   consistently do not express (under 40% of cells, all data sets): CD3D, CD3E, TRAC, TRBC2, CD69, IL32, IL7R, CD8A, CD8B, GZMK, CD79A, MS4A1, CTSW, KLRF1, NKG7, GZMB;
   single gene markers (high accuracy univariate predictors of monocytes vs. others): CST3, TYROBP, CD4;
T cells (both CD8+ and CD8−):
   consistently express (over 40% of cells, all data sets): CD3D, CD3E, IL32; all data sets except SORT for CD8+: TRAC, TRBC2, CD69, IL7R;
   consistently do not express (under 40% of cells, all data sets): CD79A, MS4A1, CST3, CD4, TYROBP, CTSW, KLRF1, GZMB;
   single gene markers (high accuracy univariate predictors of T cells vs. others): CD3E, CD3D, TRAC, IL32, IL7R, CD69; CD8+ T cells:
   consistently express (over 40% of cells, all data sets): CD3D, CD3E, IL32, CD8B; all data sets except SORT: TRAC, TRBC2, CD69, IL7R, CD8A, GZMK, NKG7;
   consistently do not express (under 40% of cells, all data sets): CD79A, MS4A1, CST3, CD4, TYROBP, CTSW, KLRF1, GZMB; single gene markers (high accuracy univariate predictors of CD8+T cells vs. non-CD8+ T cells): CD8A, CD8B, CSTW, NKG7, GZMK, GZMB, CD4;
CD8− T cells:
   consistently express (over 40% of cells, all data sets): CD3D, CD3E, TRAC, TRBC2, CD69, IL32, IL7R;
   consistently do not express (under 40% of cells, all data sets): CD8A, CD8B, GZMK, CD79A, MS4A1, CST3, CD4, TYROBP, CTSW, KLRF1, NKG7, GZMB;
   single gene markers (high accuracy univariate predictors of CD8−T cells vs. CD8+ T cells): see CD8+ T cells.

The multiclass prediction accuracy of subsets of the AMASC gene panel was also evaluated by running 10 XGBoost models for each data set, using a random selection of genes from the AMASC panel, between 1 and 19 genes. The results of this analysis are shown in Table 4 (median accuracy over 10 random selections). This analysis shows that the multiclass prediction accuracy does not excess 50% on average for fewer than 4 randomly selected predictor genes. This is not unexpected considering that the ability to correctly classify cells between 6 different cell types classes (5 different cell types and an “other cell types” classes) is being assessed, which is not possible with e.g. only one marker and Boolean levels of expression. Indeed, a single marker selected from the panel would be sufficient to accurately classify cells between two subpopulations: one that expresses the marker and one that does not. If the marker is a single gene marker then one of these subpopulation may correspond to a single cell type. However, the other subpopulation would comprise a mixture of cell types. Using a pair of markers, it becomes in theory possible to discriminate between 4 different subpopulations, provided that these have different patterns of expression of the two markers (i.e. 0/0, 1/0, 0/1, 1/1). Using a trio of markers, it becomes in theory possible to discriminate between 8 different subpopulations, provided that these have different patterns of expression of the three markers (i.e. 0/0/0, 0/1/0, 0/0/1, 0/1/1, 1/0/0, 1/0/1, 1/1/0, 1/1/1). As the skilled person understands, at least some combinations of three markers in the gene panel can be expected to discriminate between 6 subpopulations of cells with high accuracy. All combinations of markers in the gene panel, no matter their size, can be expected to discriminate between at least two subpopulations of cell types. In other words, a gene marker or gene marker combination that has relatively low accuracy in discriminating between more than two classes will still have very high accuracy in predicting whether a cell belongs to a particular cell type or not (as shown in Table 2). Further, the data in Table 4 shows that multi-class accuracy levels (for 6 classes corresponding to 5 specific cell types—NK cells, monocytes, B lymphocytes, CD8+T cells, non-CD8+ T cells—and an “other cell types” category) above 50% for all data sets can be expected with as few as 4 genes. Further, multi-class accuracy levels above 70% for all data sets obtained by multimodalities single cell sequencing (i.e. all but the PBMCSort data set) can be expected with as few as 5 genes, multi-class accuracy levels above 70% for all data sets can be expected with as few as 7 genes, and multi-class accuracy levels above 80% for all data sets can be expected with as few as 10 genes. As the skilled person understands, even better accuracies with fewer genes can be expected in relation to classifications with fewer classes, asvdemonstrated for even single genes in Table 2 (where 2 classes are used, i.e. one cell type vs all others).

TABLE 4
Multi-class accuracy for boosted tree models trained using random subsets
of the AMASC genes (median accuracy over 10 random selections).

| Number of | | | | | | | |
|---|---|---|---|---|---|---|---|
| Genes | PBMC10KV3 | PBMC10KNG | CBMC | PBMC1K | PBMC5K | MALT | SORT |
| 1 | 0.43 | 0.42 | 0.48 | 0.46 | 0.51 | 0.29 | 0.50 |
| 2 | 0.63 | 0.60 | 0.63 | 0.59 | 0.59 | 0.24 | 0.44 |
| 3 | 0.66 | 0.64 | 0.69 | 0.64 | 0.64 | 0.33 | 0.46 |
| 4 | 0.71 | 0.74 | 0.69 | 0.69 | 0.65 | 0.79 | 0.54 |
| 5 | 0.75 | 0.73 | 0.73 | 0.74 | 0.71 | 0.78 | 0.56 |
| 6 | 0.78 | 0.77 | 0.76 | 0.78 | 0.72 | 0.80 | 0.62 |
| 7 | 0.81 | 0.78 | 0.79 | 0.80 | 0.76 | 0.83 | 0.71 |
| 8 | 0.83 | 0.80 | 0.80 | 0.80 | 0.81 | 0.84 | 0.74 |
| 9 | 0.86 | 0.83 | 0.83 | 0.84 | 0.80 | 0.87 | 0.77 |
| 10 | 0.88 | 0.85 | 0.84 | 0.85 | 0.84 | 0.89 | 0.89 |
| 11 | 0.89 | 0.85 | 0.85 | 0.85 | 0.82 | 0.88 | 0.86 |
| 12 | 0.89 | 0.86 | 0.86 | 0.87 | 0.83 | 0.87 | 0.82 |
| 13 | 0.90 | 0.87 | 0.87 | 0.88 | 0.85 | 0.91 | 0.88 |
| 14 | 0.90 | 0.88 | 0.88 | 0.87 | 0.84 | 0.91 | 0.86 |
| 15 | 0.90 | 0.89 | 0.89 | 0.89 | 0.85 | 0.92 | 0.84 |
| 16 | 0.91 | 0.89 | 0.89 | 0.89 | 0.85 | 0.93 | 0.91 |
| 17 | 0.92 | 0.90 | 0.90 | 0.90 | 0.86 | 0.93 | 0.90 |
| 18 | 0.92 | 0.90 | 0.90 | 0.90 | 0.87 | 0.93 | 0.92 |
| 19 | 0.92 | 0.90 | 0.91 | 0.90 | 0.87 | 0.94 | 0.93 |

Together, the data in FIGS. 2 and 3 and Tables 2-4 shows that gene expression of genes in a succinct panel are highly predictive of the cell type of a cell, from scRNA expression data. Further, the data shows that each of these genes is able to identify at least one particular cell type with confidence (one vs. all others), as demonstrated in FIG. 3 and Table 2. The data in Tables 2 and 3 further show that subsets of this succinct panel can be constructed which can identify two or more cell types (and up to all cell types) with confidence. For example, the data shows that the genes CST3, CD8B, IL7R, KLRF1 and MS4A1 are each able to identify a particular cell type with high confidence (see FIG. 3), together forming a set that is sufficient to identify each of the five cell types that each of these gene is a high confidence univariate predictor of. The data in FIGS. 2 and 3 further indicates that in this subset, CD8B could be replaced or complemented with CD8A, and IL7R could be replaced or complemented with TRBC2 and/or CD3D with no or minimal loss of accuracy (in the case of a replacement). Similarly, as indicated by the data in FIG. 3, a good accuracy of cell type assignment could still be achieved by replacing CST3 with TYROBP, CD8B with any of CSTW, NKG7, GZMK, GZMB, and CD4, IL7R with any of CD3E, TRAC, IL32, and CD69, KLRF1 with any of NKG7, CTSW, and GZMB, and MS4A1 with CD79A. Further, for the purpose of identifying fewer than all of the 6 different cell types classes (5 specific cell types and “others”), smaller subsets of genes can be constructed which would perform the task with high accuracy. For example, CTSW (or GZMB or TYROBP or KLRF1) alone can be used to identify with confidence whether a cell is a NK cell or not. A combination of CTSW (or GZMB or TYROBP or KLRF1) and CD79A (or MS4A1) can be used to identify with confidence whether a cell is a NK cell, a B cell or another type of cell. A combination of CTSW (or GZMB or TYROBP or KLRF1) and CD79A (or MS4A1) and CST3 (or CD4) can be used to identify with confidence whether a cell is a NK cell, a B cell, a monocyte, a T cell or another type of cell. A combination of CTSW (or GZMB or TYROBP or KLRF1) and CD79A (or MS4A1) and CST3 (or CD4) and IL7R (or any of CD3D, CD3E, TRAC, TRBC2, IL32, CD69) can be used to identify with confidence whether a cell is a NK cell, a B cell, a monocyte, a CD8+ T cell, a CD8− T cell or another type of cell. In short, any (even random) combination (of any size) of the genes in the AMASC panel can be used to identify at least one cell type, multiple selected combinations of the genes in the AMASC panel can be used to discriminate between two or more cell types, and any random combination of at least 10 genes can be expected to discriminate between at least five cell types with high confidence.

Of note, seven of the selected genes encode proteins commonly used as part of panels of immunostaining surface markers, i.e. CD79A, CD3D, CD3E, CD8A, CD8B, CD4, and MS4A1 (Jerby-Arnon et al., 2018; Karaayvaz et al., 2018; Lambrechts et al., 2018; Puram et al., 2017). However, some of the genes that encode commonly used cell-type markers such as NCAM1 (encodes CD56) and CD19 were not among the selected genes. This underlines the observation that there is a weak correlation between protein and mRNA expressions in the multi-modal data. In other words, it is not the case that a protein being a reliable cell type marker necessarily implies that the gene expression of the protein (i.e. the transcript level data measured e.g. by scRNA sequencing) would be a reliable (or even remotely predictive) cell type marker. The process selected KLRF1, CD79A and MS4A1 for natural killers and B lymphocytes, respectively, which are more predictive of the cell types than NCAM1 and CD19 (see FIG. 3). The correlation of the expression of the selected genes and the cell types is not limited to one-to-one correspondence. For example, TYROBP is expressed by monocytes and natural killer cells, and NKG7 is expressed by natural killers and CD8+ T cells. The process also reveals that there are genes that do not encode surface markers but are predictive of cell types, such as CST3 and IL32.

Further, the selected features are advantageously biologically interpretable. Indeed, the selected genes for T lymphocyte populations included the subunits of the CD3 protein (CD3D, CD3E), and the subunits of the CD8 protein (CD8A, CD8B), which are used as protein markers for T lymphocytes. They further included TRAC and TRBC2, which encode for the constant regions of T cell receptors (TCR), which are located on the surface of T lymphocytes and allow the T lymphocytes to recognize antigens through peptide binding. The process also identified IL32 as a marker of T lymphocytes and natural killers. This gene encodes pro-inflammatory cytokine interleukin 32, which induces monocytes to produce inflammatory cytokines and chemokines (Kim et al., 2005).

Turning now to B cells, although CD19 is often used as a marker of the B cell lineage (Adams et al., 2009), the process instead selected CD79A and MS4A1, which were more predictive than CD19 as the B cell markers in the scRNA-seq data.

The genes that are related to cytotoxicity (KLRF1, NKG7, GZMB, and CTSW, which encode killer cell lectin like receptor F1, natural killer cell granule protein 7, granzyme B, and cathepsin W, respectively) were all selected as markers of natural killer cells (NK), while NCAM1 (which encodes CD56) was not selected. NKG7, CTSW, GZMB are known to be expressed in the natural killer cells and cytotoxic T cells, while KLRF1 is expressed in natural killer cells (Bezman et al., 2012; Biassoni et al., 2001; Turman et al., 1993; Wex et al., 2001).

For monocytes or macrophages, CST3 (encodes cystatin C) was selected as the cell-type marker with the highest importance in the model (FIG. 2). The protein of CST3 is a protease inhibitor expressed in monocytes (Gren et al., 2015). Interestingly, the process identified genes, such as TYROBP (TYRO protein tyrosine kinase binding protein), that were expressed in both monocytes and natural killer cells. The two cell types belong to different cell lineages, but the gene indeed has been reported to be produced by myeloid and lymphoid cells and is involved in activating inflammatory response (Bakker et al., 1999; Kiialainen et al., 2005).

### EXAMPLE 2

### Classification Models Based on the AMASC Panel Accurately and Efficiently Assign Cell Types Across ScRNAseq Data Sets

In this example, the inventors used the validation data as described above to demonstrate that the panel of mRNA markers for cell type assignment identified as outlined in Example 1 can be used to accurately and efficiently assign cell types in scRNAseq data with a variety of classification algorithms trained using the training data described above.

### Methodology

The three processed training data sets were concatenated into one training set, which was filtered to only include the expression data for the 19 genes selected as described in Example 1 (see FIG. 1, step 160). This data was used to train a variety of multiclass classifiers (see FIG. 1, step 170), including: a boosted tree classifier (as implemented in XGBoost, setting “objective=multi:softmax”), a support vector machine classifier (SVM, as implemented in scikit-learn (Pedregosa et al., 2011)—function LinearSVC), and a logistic regression classifier (as implemented in scikit-learn, function LogisticRegression). The SVM multiclass classifier was Implemented as a set of binary one-vs-rest classifiers (each classifier predicting the probability that a cell belongs to one of the cell type class vs. all other cell type classes), the output of which is combined into a single class prediction by selecting the highest probability prediction for a cell across binary classifiers. The logistic regression classifier was implemented as a multi-class classifier using a multinomial distribution to fit the data. All classifiers were trained using the default parameters in the respective implementations. The trained models were then evaluated using the accuracy function in scikit-learn package, on the validation sets. This function computes the fraction of samples correctly predicted (i.e. TP+TN/(TP+TN+FP+FN), where FN and FP refer to false negatives and false positives, respectively) for each subset (i.e. for each type of cells).

The trained models were also compared with published methods for predicting cell types from scRNAseq data. In particular, the following methods were used: CaSTLe, SingleR, and CellAssign. For SingleR and CellAssign, the training data and parameters provided in the original publications were used to train the models. For SingleR, two versions were tested, using different reference datasets: HPCA (Human Primary Cell Atlas, Mabbott et al. 2013)and Encode (The ENCODE Project Consortium 2012). For CellAssign, two versions were also tested, on using the default gene panel (referred to as “TME”), and one using the default gene panel supplemented with the AMASC gene panel (referred to as “TME+AMASC”). As CaSTLe did not have a corresponding immune cell dataset in the original publication, the model was trained using the training data described above (using binarised values for all 3 training data sets concatenated), and the class labels above. Since the other published methods used different sets of cell type labels, the labels of cell types provided by each model were manually mapped to the 6 categories described above, i.e. non-CD8+ T lymphocytes, CD8+ T lymphocytes, B lymphocytes, natural killer cells, monocytes and other cells, in order to benchmark the performance of the various methods. In particular, CellAssign (TME) uses the following categories: B cells, T cells, Cytotoxic T cells, Monocyte/Macrophage, Epithelial cells, Myofibroblast, Vascular smooth muscle cells, Endothelial cells, other. Cytotoxic T cells were mapped to “CD8+ T cells”; T cells were mapped to “non-CD8+T cells”; the non-immune cells were mapped to “other cells”. For CellAssign (TME+AMASC), the NK cells class was added. Similarly, for SingleR (Encode) and SingleR (HPCA), the following subpopulations mapping were used: CD8+ T cells were mapped to “CD8+ T cells” and the rest of T cells to “non-CD8+ T cells”; Monocytes, Macrophages, Neutrophils were mapped to “monocytes”; B cell subpopulations and plasma were mapped to “B lymphocytes”; NK were mapped to “natural killer cells”; all other categories were mapped to “other cells”. The methods were compared in terms of cell type prediction accuracy and computational time for obtaining a prediction (time/cell) using an Intel® Xeon® E5-2680 v3 processor.

### Results

Using independent validation data sets, the inventors compared the performances of the models trained on the AMASC panel with those of published methods, including SingleR (Aran et al., 2019), CaSTLe (Lieberman et al., 2018), and CellAssign (A. W. Zhang et al., 2019), which are top performing machine learning models among immune cell assignment methods (Abdelaal et al., 2019). As explained above, SingleR uses the correlation between the expression profile of a cell and the expression profied of purified cells in reference data sets (including bulk RNA sequencing datat sets) to identify the most likely cell type of the cell. CaSTLe applies a feature selection then classification process using published scRNAseq data sets with manual annotation of clusters of cells as ground truth. For the feature selection step, CaSTle uses a combination of expression level (choosing highly expressed genes across the source/reference data set and the target/validation dataset) and mutual information between expression and cell type in the source data set (genes that are highly associated with the ground truth class labels). CellAssign uses a probabilistic graphical model to probabilistically assign each cell to a given cell type, using raw count data from a heterogeneous scRNA-seq population, along with a set of known marker genes for various cell types under study. The marker genes can be provided as the result of expert manual curation, or are identified based on differential expression between cell types (using data from different known cell types including mostly bulk RNA sequencing data).

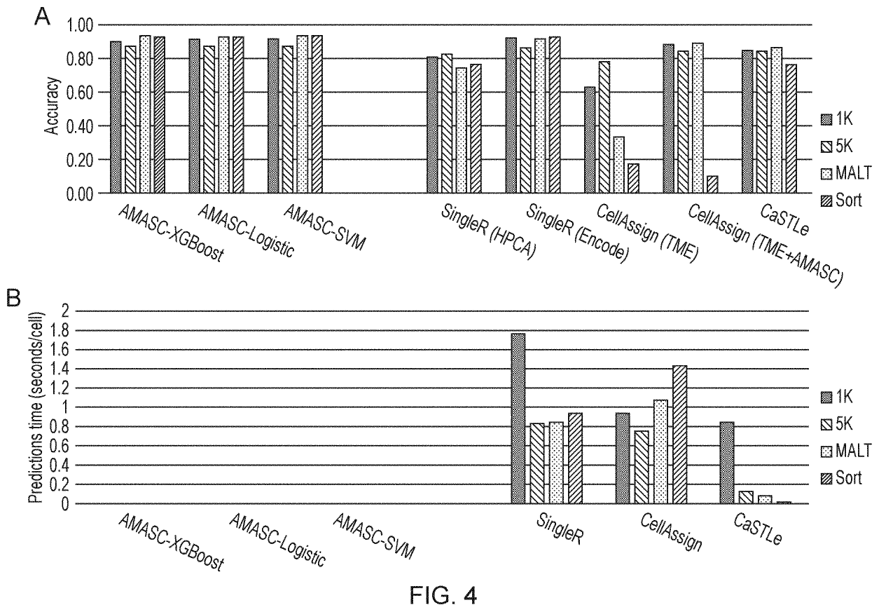

The accuracy of the machine learning models based on the AMASC panel (AMASC-XGBoost, AMASC-Logistic, AMASC-SVM) were above 0.84 and up to 0.94 for the analyzed data sets (see FIG. 4A). The high accuracies observed across all of the models (i.e. regardless of the particular type of classifier used) and all of the validation data sets suggest that the genes in the identified panel are agnostic to the models, and that they are likely generalizable cell type predictors across different data sets, tissues of origin, and platforms. Indeed, the validation data sets included data from a tissue of origin that was not represented in the training set (MALT), and from a data acquisition platform that was not represented in the training set (FAC-sorted cell sequencing). The models trained using the AMASC panel outperformed the comparative published methods on most of the test data sets. SingleR (Encode) slightly outperformed the AMASC models on the PBMC1K data set. In particular, the AMASC model misclassified more CD8⁺ T cells as non-CD8⁺ T cells than SingleR (Encode) for this particular data set. The difference in accuracy was very small (0.911 vs 0.920) especially considering the vastly different sizes of the predictor sets (19-genes for the AMASC models vs whole-transcriptome for SingleR), and the AMASC models had similar or higher accuracies than the SingleR models for all other data sets. The CellAssign model uses a marker panel that does not have a label for natural killer cells (A. W. Zhang et al., 2019), which impacted its performance since the natural killer cells could not be accurately classified. The original CellAssign model is not predictive on the non-PBMC data sets and the FAC-sorted dataset. Replacing the immune genes in its panel with the AMASC panel improved the performance of CellAssign on all the datasets except for the FAC-sorted data set.

The inventors further benchmarked the runtime of the AMASC-based models against other popular tools. The median (across all data sets) runtimes of the trained AMASC XGBoost, logistic, and SVM models were 4.08, 0.13, and 0.7 milliseconds (see FIG. 4B). The median (across all data sets) runtimes of SingleR, CellAssign, and CaSTLe for annotating a (single) cell are 844.75, 955.45, and 87.94 milliseconds (for the validation data sets the median run times were 894.96, 1006.59, 108.83 ms, respectively). The AMASC models ran up to 700 times faster than the other methods with the task of cell-type assignment.

### Discussion

In this study, the inventors developed a method to identify cell-type predictive genes using the single-cell multi-modal sequencing data and machine learning, which does not rely on the clustering of cells based on gene expression data or the similarities between cells derived from a large number of genes. Using this approach, they identified a small set of genes and demonstrated that these were sufficient to train machine-learning models that accurately predict the types of immune cells in human blood.

Since these genes were discovered using the mRNA expression and the protein-based phenotype of the same cell, the can be expected perform better at predicting cell types than features selected either by known protein-based markers or by differential gene expression analysis using the RNA-seq data alone. The comparison with the published cell-type assignment methods shows that the machine learning models based only on the AMASC panel outperformed the published methods that rely on clustering and either the empirically selected markers (CellAssign), univariate-feature selection (CaSTLe), or the resemblance to known expression profiles in bulk RNA-seq databases (SingleR) in most of the test data sets.

Since the three AMASC-based models performed equally well on the test sets, it is reasonable to assume that the proposed gene panel is agnostic to the different mechanisms of machine learning models, and likely generalizable across data sets. When this panel was used to replace the original marker genes in other published methods (in particular, CellAssign), improvements in the performance of the model for cell-type assignment were also observed.

It is interesting that many of the individual genes in this panel are more accurate in predicting cell types than the commonly used cell-type markers, for example, CD79A and MS4A1 for B lymphocytes, CST3 for monocytes, and KLRF1 for natural killer cells. In addition to confirming the known cell type markers, past studies support the roles of these selected genes in the molecular physiology of the corresponding cell populations. The association of the genes and the cell types are not exclusive, however. For example, NKG7 and CTSW are associated with both cytotoxic T cells and natural killer cells. The CD3 subunits, T cell receptor subunits, IL32 and IL7R are all associated with T cells, which suggest that although the AMASC panel is compact in terms of the number of features, it retains some feature redundancy. This indicates that the full panel is likely not necessary to obtain an accurate assignment. However, redundancy in the panel by including more (or all of the genes) would improve the panel's ability to deal with uncertainty in the data. A parsimonious selection of features offers not only high interpretability of the models but also low computational complexity for training a model and making predictions. This is especially useful in its practical use.

Since the AMASC panel consists of fewer than 20 genes, the AMASC models are more than 700 times faster than the other methods compared in the study on annotating cells while achieving a superior accuracy.

Finally, because the genes in the AMASC panel are closely associated with the molecular characteristics of the immune cells, it is reasonable to expect that they may also be applicable in predicting immune cell types in many tissue types, including diseases beyond hematologic cancers. For example, the AMASC panel is likely to have some applicability in tissue types other than PBMC, CBMC and MALT, and in identifying subpopulations (e.g., naive CD8⁺ T cells) of immune cells. In particular, the AMASC panel is likely to have some applicability in any tissue that is not immune deserted. The AMASC panel is further likely to have applicability in any tissue that has an immune microenvironment that has a cell type composition (at least in terms of which types of immune cells are present) similar to that of PBMC, CBMC and MALT tissues.

### REFERENCES

Abdelaal, T., et al. (2019). A comparison of automatic cell identification methods for single-cell RNA sequencing data. Genome Biol, 20(1), 194. doi:10.1186/s13059-019-1795-z
Adams, H., et al. (2009). Diagnostic utility of the B-cell lineage markers CD20, CD79a, PAX5, and CD19 in paraffin-embedded tissues from lymphoid neoplasms. Appl Immunohistochem Mol Morphol, 17(2), 96-101. doi:10.1097/PAI.0b013e3181845ef4
Aran, D., et al. (2019). Reference-based analysis of lung single-cell sequencing reveals a transitional profibrotic macrophage. Nat Immunol, 20(2), 163-172. doi:10.1038/541590-018-0276-y
Bakker, A. B., et al. (1999). Myeloid DAP12-associating lectin (MDL)-1 is a cell surface receptor involved in the activation of myeloid cells. Proc Natl Acad Sci U S A, 96(17), 9792-9796. doi:10.1073/pnas.96.17.9792
Becht, E., et al. (2019). Dimensionality reduction for visualizing single-cell data using UMAP. Nature Biotechnology, 37(1), 38-+. doi:10.1038/nbt.4314
Bezman, N. A., et al. (2012). Molecular definition of the identity and activation of natural killer cells. Nat Immunol, 13(10), 1000-1009. doi:10.1038/ni.2395
Biassoni, R., et al. (2001). Human natural killer cell receptors and co-receptors. Immunol Rev, 181, 203-214. doi:10.1034/j.1600-065x.2001.1810117.x
Domanskyi, S., et al. (2019). Polled Digital Cell Sorter (p-DCS): Automatic identification of hematological cell types from single cell RNA-sequencing clusters. BMC Bioinformatics, 20(1), 369. doi:10.1186/s12859-019-2951-x
Gren, S. T., et al. (2015). A Single-Cell Gene-Expression Profile Reveals Inter-Cellular Heterogeneity within Human Monocyte Subsets. PLoS One, 10(12). doi:ARTN e0144351
10 10.1371/journal.pone.0144351
Hashimshony T, Wagner F, Sher N, Yanai I (September 2012). “CEL-Seq: single-cell RNA-Seq by multiplexed linear amplification”. Cell Reports. 2 (3): 666-73. doi:10.1016/j.celrep.2012.08.003
Heath, J. R., et al. (2016). Single-cell analysis tools for drug discovery and development. Nat Rev Drug Discov, 15(3), 204-216. doi:10.1038/nrd.2015.16
Islam S, Kjallquist U, Moliner A, Zajac P, Fan J B, Lonnerberg P, Linnarsson S (July 2011). “Characterization of the single-cell transcriptional landscape by highly multiplex RNA-seq”. Genome Research. 21 (7): 1160-7. doi:10.1101/gr.110882.110
Jerby-Arnon, L., et al. (2018). A Cancer Cell Program Promotes T Cell Exclusion and Resistance to Checkpoint Blockade. Cell, 175(4), 984-997 e924. doi:10.1016/j.cell.2018.09.006
Karaayvaz, M., et al. (2018). Unravelling subclonal heterogeneity and aggressive disease states in TNBC through single-cell RNA-seq. Nature Communications, 9(1), 3588. doi:10.1038/s41467-018-06052-0
Kiialainen, A., et al. (2005). Dap12 and Trem2, molecules involved in innate immunity and neurodegeneration, are co-expressed in the CNS. Neurobiol Dis, 18(2), 314-322. doi:10.1016/j.nbd.2004.09.007
Kim, S. H., et al. (2005). Interleukin-32: a cytokine and inducer of TNFalpha. Immunity, 22(1), 131-142. doi:10.1016/j.immuni.2004.12.003
Kiselev, V. Y., et al. (2019). Challenges in unsupervised clustering of single-cell RNA-seq data. Nat Rev Genet. doi:10.1038/s41576-018-0088-9 Kouno T, Moody J, Kwon AT, Shibayama Y, Kato S, Huang Y, et al. (January 2019). “Cl CAGE detects transcription start sites and enhancer activity at single-cell resolution”. Nature Communications. 10 (1): 360. Bibcode:2019NatCo . . . 10 . . . 360K. doi:10.1038/s41467-018-08126-5
Lambrechts, D., et al. (2018). Phenotype molding of stromal cells in the lung tumor microenvironment. Nat Med, 24(8), 1277-1289. doi:10.1038/s41591-018-0096-5
Levine, J. H., et al. (2015). Data-Driven Phenotypic Dissection of AML Reveals Progenitor-like Cells that Correlate with Prognosis. Cell, 162(1), 184-197. doi:10.1016/j.cell.2015.05.047
Lieberman, Y., et al. (2018). CaSTLe—Classification of single cells by transfer learning: Harnessing the power of publicly available single cell RNA sequencing experiments to annotate new experiments. PLoS One, 13(10), e0205499. doi:10.1371/journal.pone.0205499
Lun, A. T., et al. (2016). A step-by-step workflow for low-level analysis of single-cell RNA-seq data with Bioconductor. F1000Res, 5, 2122. doi:10.12688/f1000research.9501.2
Ma, F., and Pellegrini, M. (2019). ACTINN: Automated Identification of Cell Types in Single Cell RNA Sequencing. Bioinformatics. doi:10.1093/bioinformatics/btz592
Mabbott, Neil A., J. K. Baillie, Helen Brown, Tom C. Freeman, and David A. Hume. 2013. “An expression atlas of human primary cells: Inference of gene function from coexpression networks.” BMC Genomics 14. doi:10.1186/1471-2164-14-632
Macosko et al., (2015) Highly Parallel Genome-wide Expression Profiling of Individual Cells Using Nanoliter Droplets. Cell, Volume 161, Issue 5, 21 May 2015, Pages 1187-1201. doi.org:10.1016/j.cell.2015.05.002
Pedregosa, F. et al. (2011). Scikit-learn: Machine Learning in Python. Journal of Machine Learning Research, volume 12, pages 2825--2830.
Peterson, V. M., et al. (2017). Multiplexed quantification of proteins and transcripts in single cells. Nature Biotechnology, 35(10), 936-939. doi:10.1038/nbt.3973
Puram, S. V., et al. (2017). Single-Cell Transcriptomic Analysis of Primary and Metastatic Tumor Ecosystems in Head and Neck Cancer. Cell, 171(7), 1611-1624 e1624. doi:10.1016/j.cell.2017.10.044
Ramskold D, Luo S, Wang Y C, Li R, Deng Q, Faridani O R, et al. (August 2012). “Full-length mRNA-Seq from single-cell levels of RNA and individual circulating tumor cells”. Nature Biotechnology. 30 (8): 777-82. doi:10.1038/nbt.2282
Xavier Robin, Natacha Turck, Alexandre Hainard, Natalia Tiberti, Frédérique Lisacek, Jean-Charles Sanchez and Markus Müller (2011). “pROC: an open-source package for R and S+ to analyze and compare ROC curves”. BMC Bioinformatics, 12, p. 77. DOI: 10.1186/1471-2105-12-77
Sasagawa Y, Nikaido I, Hayashi T, Danno H, Uno K D, Imai T, Ueda H R (April 2013). “Quartz-Seq: a highly reproducible and sensitive single-cell RNA sequencing method, reveals non-genetic gene-expression heterogeneity”. Genome Biology. 14 (4): R31. doi:10.1186/gb-2013-14-4-r31
Singh M, Al-Eryani G, Carswell S, Ferguson J M, Blackburn J, Barton K, et al. (July 2019). “High-throughput targeted long-read single cell sequencing reveals the clonal and transcriptional landscape of lymphocytes”. Nature Communications. 10 (1): 3120. Bibcode:2019NatCo . . . 10.3120S. doi:10.1038/s41467-019-11049-4
Stoeckius, M., et al. (2017). Simultaneous epitope and transcriptome measurement in single cells. Nat Methods, 14(9), 865-868. doi:10.1038/nmeth.4380
Tang F, Barbacioru C, Wang Y, Nordman E, Lee C, Xu N, et al. (May 2009). “mRNA-Seq whole-transcriptome analysis of a single cell”. Nature Methods. 6 (5): 377-82. doi:10.1038/NMETH.1315
The ENCODE Project Consortium. 2012. “An integrated encyclopedia of DNA elements in the human genome.” Nature. doi:10.1038/nature11247.
Turman, M. A., et al. (1993). Characterization of a Novel Gene (Nkg7) on Human Chromosome-19 That Is Expressed in Natural-Killer-Cells and T-Cells. Human Immunology, 36(1), 34-40. doi:Doi 10.1016/0198-8859(93)90006-M
Wex, T., et al. (2001). Human cathepsin W, a cysteine protease predominantly expressed in NK cells, is mainly localized in the endoplasmic reticulum. Journal of Immunology, 167(4), 2172-2178. doi:DOI 10.4049/jimmunol.167.4.2172
Xie, P., et al. (2019). SuperCT: a supervised-learning framework for enhanced characterization of single-cell transcriptomic profiles. Nucleic Acids Res, 47(8), e48. doi:10.1093/nar/gkz116
Zhang, A. W., et al. (2019). Probabilistic cell-type assignment of single-cell RNA-seq for tumor microenvironment profiling. Nat Methods, 16(10), 1007-1015. doi:10.1038/s41592-019-0529-1
Zhang, X., et al. (2019). CellMarker: a manually curated resource of cell markers in human and mouse. Nucleic Acids Res, 47(D1), D721-D728. doi:10.1093/nar/gky900
Zheng, G. X., et al. (2017). Massively parallel digital transcriptional profiling of single cells. Nat Commun, 8, 14049. doi:10.1038/ncomms14049

All references cited herein are incorporated herein by reference in their entirety and for all purposes to the same extent as if each individual publication or patent or patent application was specifically and individually indicated to be incorporated by reference in its entirety. The specific embodiments described herein are offered by way of example, not by way of limitation. Any sub-titles herein are included for convenience only, and are not to be construed as limiting the disclosure in any way.

1. A method of identifying a predictor set of genes for predicting the cell type of one or more cells, the method comprising:
(a) obtaining a single cell gene expression profile comprising gene expression measurements for a set of genes, for a plurality of cells, and a single cell protein expression profile comprising protein expression measurements for two or more proteins, for the plurality of cells;
(b) using the single cell protein expression profiles and an unsupervised learning method to assign a cell type class to at least some of the plurality of cells;
(c) scaling or discretising the measurements for each gene in the single cell gene expression profiles;
(d) applying a feature selection process to the single gene expression profiles to identify genes in the single cell gene expression profiles that are predictive of the cell type classes assigned in step (b), wherein the genes identified in step (d) form a predictor set of genes for predicting the cell type of one or more cells.

2. The method of claim 1, wherein the unsupervised learning method is a clustering method, wherein using the single cell protein expression profiles and an unsupervised learning method to assign a cell type class to at least some of the plurality of cells comprises clustering the single cell protein expression profiles to identify at least a first group of cells and a second group of cells, and assigning a common cell type class to cells in at least one of the groups using the protein expression profiles of the cells in said group.

3. The method of claim 1, wherein the predictor set of genes comprises at most at most 100 genes, at most 90 genes, at most 80 genes, at most 70 genes, at most 60 genes, at most 50 genes, at most 40 genes, at most 30 genes, or at most 20 genes; and/or wherein the predictor set of genes when used as predictive features of a classification algorithm results in a classification algorithm that predicts cell types corresponding to the cell type classes of step (b) with an accuracy of at least 70%, at least 80% or at least 90%, using the single cell gene expression profiles obtained at step (a).

4. The method of claim 1, comprising scaling the measurements for each gene in the single cell gene expression profiles between 0 and 1, wherein scaling comprises a linear mapping of the range of minimum to maximum expression for each gene to the [0,1] interval.

5. The method of claim 1, comprising binarising the measurements for each gene in the single cell gene expression profiles.

6. The method of claim 1, comprising log transforming the measurements for each protein in the single cell protein expression profile, prior to applying the unsupervised learning method to assign a cell type class to at least some of the plurality of cells.

7. The method of claim 1, comprising binarising the measurements for each protein in the single cell protein expression profile.

8. The method of claim 1, wherein applying a feature selection process comprises training one or more classifiers to predict the cell type classes assigned in step (b) using the single gene expression profiles as input variables.

9. The method of claim 8, wherein applying a feature selection process comprises training a plurality of classifiers to predict the cell type classes assigned in step (b) using the single gene expression profiles as input variables, and identifying genes in the single gene expression profiles that are predictive of the cell type classes assigned in step (b) comprises comparing the genes used by the plurality of classifiers in making a prediction and selecting those genes that are used by two or more of the plurality of classifiers.

10. The method of claim 1, wherein obtaining a single cell gene expression profile and a single cell protein expression profile for a plurality of cells comprises obtaining a single cell gene expression profile and a single cell protein expression profile for a first plurality of cells, and for at least a further plurality of cells, wherein the expression profiles for the first and further plurality of cells were obtained through separate experiments, preferably wherein steps (b), (c) and/or (d) are performed independently for the first and further plurality of cells.

11. The method of claim 10, wherein the feature selection process of step (d) is performed independently for the first and further plurality of cells, and genes in the single gene expression profiles that are predictive of the cell type classes assigned in step (b) are identified based on the combined outputs of the independent feature selection processes.

12. The method of claim 10, wherein the first and one or more further plurality of cells have been obtained from at least two different types of samples, where the samples may differ by e.g. their tissue of origin, and/or wherein the expression profiles for the first and one or more further plurality of cells have been obtained using at least two different experimental protocols.

13. The ding claim claim 1, wherein steps (b) to (d) are computer implemented, and/or wherein step (a) comprises processing one or more samples of cells or tissues using a combined single cell transcriptomics and proteomics protocol, or wherein step (a) is computer-implemented and comprises receiving a previously acquired single cell gene expression profile and a previously acquired single cell protein expression profile for the plurality of cells.

14. A method for predicting the cell type of one or more cells, the method comprising:
(i) obtaining a predictor set of genes, wherein the predictor set of genes has been identified using the method of claim 1; optionally wherein obtaining a predictor set of genes comprises identifying a predictor set of genes using the method of claim 1;
(ii) obtaining a single cell gene expression profile for each of the one or more cells comprising gene expression measurements for a set of genes comprising at least 1, 2, 3, 4, 5, 6, 7 or more (such as all of) the predictor set of genes; and
(iii) making a prediction of the cell type of the one or more cells based at least in part on the gene expression profile for the predictor set of genes.

15. A system comprising:
at least one processor; and
at least one non-transitory computer readable medium containing instructions that, when executed by the at least one processor, cause the at least one processor to:
(a) obtain a single cell gene expression profile comprising gene expression measurements for a set of genes, for a plurality of cells, and a single cell protein expression profile comprising protein expression measurements for two or more proteins, for the plurality of cells;
(b) use the single cell protein expression profiles and an unsupervised learning method to assign a cell type class to at least some of the plurality of cells;
(c) scale or discretise the measurements for each gene in the single cell gene expression profiles;
(d) apply a feature selection process to the single gene expression profiles to identify genes in the single cell gene expression profiles that are predictive of the cell type classes assigned in step (b), wherein the genes identified in step (d) form a predictor set of genes for predicting the cell type of one or more cells.
